(19) United States
(12) Patent Application Publication
(10) Pub. No.: US 2023/0397634 A1
(43) Pub. Date: Dec. 14, 2023

(54) METHODS AND SYSTEMS FOR USING HYDROPONICALLY SPROUTED CEREAL GRAINS FOR IMPROVING SKELETAL DEVELOPMENT IN PREPUBESCENT RUMINANTS

(71) Applicant: HydroGreen, Inc., Langley (CA)

(72) Inventor: Shawn Jenkins, Colton, SD (US)

(73) Assignee: HydroGreen, Inc., Langley (CA)

(21) Appl. No.: 17/838,673

(22) Filed: Jun. 13, 2022

(51) Int. Cl. A23K 10/30; A01G 22/20; A01G 31/00; A23K 20/147; A23K 20/163; A23K 20/184; A23K 50/10; A23K 50/60; A61K 36/899

(52) U.S. Cl. CPC A23K 10/30; A01G 22/20; A01G 31/00; A23K 20/147; A23K 20/163; A23K 20/184; A23K 50/10; A23K 50/60; A61K 36/899

(57) ABSTRACT

Processes and methods for hydroponically sprouted cereal grains are disclosed as a mechanism for improving skeletal development, increasing nutrient levels, and maintaining or increasing future milk yield in ruminants by administering to a ruminant a feed ration having at least one feed component comprising one or more hydroponically sprouted cereal grains.

### FIELD OF THE DISCLOSURE

The present disclosure relates to skeletal development in prepubescent ruminants. More particularly, but not exclusively, the present disclosure relates to methods and systems for using hydroponically sprouted cereal grains for improving skeletal development in calves.

### BACKGROUND

Increases in skeletal size rather than body weight has shown to be the best predictor of first lactation milk yield while also showing a negative correlation to dystocia in dairy replacement heifers (Heinrichs and Hargrove, 1987; Sieber et al., 1988; Markusfeld and Ezra, 1993). With approximately 80% of skeletal growth occurring prior to puberty and 50% occurring prior to six months of age, environmental and nutritional management of replacement heifer calves has a long-term influence on a dairy's productivity (Kertz et al., 1998). Therefore, what is needed is a process, method, and system for using hydroponically sprouted cereal grains to improve skeletal developments and nutritional management.

### SUMMARY

Therefore, it is a primary object, feature, or advantage of the present disclosure to improve over the state of the art.

It is a further object, feature, or advantage of the present disclosure to improve nutritional levels in prepubescent ruminants.

It is a still further object, feature, or advantage of the present disclosure to improve skeletal development in ruminants.

Another object, feature, or advantage is to increase height and length of a ruminant.

Yet another object, feature, or advantage is to increase leptin levels in a ruminant.

Another object, feature, or advantage is to increase dry matter intake during the prepubescent period.

Yet another object, feature, or advantage is to increase insulin growth factor 1 utilizing a feed composition that includes hydroponically grown cereal grains.

Another object, feature, or advantage is to increase average daily gain in ruminants.

It is still a further object, feature, or advantage is to decrease the cost of the average daily gain in ruminants.

Yet another object, feature, or advantage is to improve the skeletal development of a ruminant while maintaining or increasing future milk yield.

In at least one aspect of the present disclosure, a method for using hydroponically sprouted cereal grains as a mechanism for improving skeletal development is disclosed. The method may include administering to a ruminant a feed ration having at least one feed component that may include one or more hydroponically sprouted cereal grains for improving skeletal developments in a ruminant.

In another aspect of the present disclosure a method for improving skeletal development in a ruminant is disclosed. The method may include hydroponically sprouting one or more cereal grains for at least one feed component for ruminants. The method may also include feeding the ruminant a feed ration having the at least one feed component that may include the one or more hydroponically sprouted cereal grains for improving skeletal development in the ruminant. The method may further include increasing insulin growth factor 1 levels in the ruminant with the feed ration. The increase in insulin growth factor 1 levels may support skeletal development within the ruminant.

In another aspect of the present disclosure a system for hydroponically sprouted cereal grains as a mechanism for improving skeletal development of a ruminant is disclosed. The system may include a grower for hydroponically sprouting one or more cereal grains for growing at least one feed component for prepubescent ruminants. The system may also include a feed ration having the at least one feed component that may include the one or more hydroponically sprouted cereal grains for improving skeletal development in the prepubescent ruminants.

One or more of these and/or other objects, features, or advantages of the present disclosure will become apparent from the specification and claims that follow. No single aspect need provide each and every object, feature, or advantage. Different aspects may have different objects, features, or advantages. Therefore, the present disclosure is not to be limited to or by any objects, features, or advantages stated herein.

### BRIEF DESCRIPTION OF THE DRAWINGS

Illustrated aspects of the disclosure are described in detail below with reference to the attached drawing figures, which are incorporated by reference herein.

FIG. 1 is an exemplary schematic illustrating enteric methanogenesis in ruminants.

FIG. 2 is an exemplary schematic illustrating carbohydrate fermentation in the rumen.

FIG. 3 is an exemplary illustration of the interaction between phytohormones and dry matter in accordance with an illustrative aspect of the disclosure.

FIG. 4A is a pictorial representation of animal feed grown under hypoxic conditions in accordance with an illustrative aspect of the present disclosure.

FIG. 4B is a pictorial representation of animal feed grown under aerobic conditions in accordance with an illustrative aspect of the present disclosure.

FIG. 5 is chart illustrating adenosine triphosphate (ATP) production under different environmental conditions in accordance with an illustrative aspect of the present disclosure.

FIG. 6 is an illustration of the interaction between phytohormones in accordance with an illustrative aspect of the disclosure.

FIG. 7 is an exemplary illustration of the hydrolysis reaction of cellulose and xylan.

FIG. 8 is an exemplary illustration depicting the hydrolysis of maltose into two glucose molecules.

FIG. 9 is an exemplary illustration depicting Adenosine Triphosphate production.

FIG. 10 is a chart illustrating a fixed effect estimate and a 95% confidence interval of starch and ethanol soluble carbohydrate or sugar concentration on a dry matter basis days post seeding in accordance with an exemplary aspect of the present disclosure.

FIG. 11 is a chart illustrating results for hydroponically sprouted test rations and control rations in accordance with an exemplary aspect of the present disclosure.

FIG. 12 is another chart illustrating hydroponically sprouted grain test ration and control ration in accordance with an exemplary aspect of the present disclosure.

FIG. 13 is another chart illustrating hydroponically sprouted grain test ration and control ration in accordance with an exemplary aspect of the present disclosure.

FIG. 14 is an additional chart illustrating hydroponically sprouted grain test ration and control ration in accordance with an exemplary aspect of the present disclosure.

FIG. 15 is a chart illustrating nutrient composition of hydroponically grown sprouts in accordance with an exemplary aspect of the present disclosure.

FIG. 16 is a chart illustrating daily dry matter intake of the hydroponically sprouted grain test ration and the control ration in ruminants in accordance with an exemplary aspect of the disclosure.

FIG. 17 is a chart illustrating reticulum pH in ruminants digesting the hydroponically sprouted grain test ration and the control ration in accordance with an exemplary aspect of the disclosure.

FIG. 18 is an exemplary illustration of the grower system in accordance with an illustrative aspect of the disclosure.

FIG. 19 is a side perspective view of a portion of the seed bed of the growing system in accordance with an illustrative aspect of the disclosure.

FIG. 20 is an end perspective view of a portion of the grower system further illustrating the seed bed thereof.

FIG. 21 is a side perspective view of a portion of the grower system illustrating another seed bed thereof.

FIG. 22 is a block diagram illustrating another perspective of the grower system in accordance with an exemplary aspect of the present disclosure.

FIG. 23 is a flowchart illustrating an exemplary process for improving skeletal development.

FIG. 24 is a flowchart illustrating another exemplary process for improving skeletal development.

### BRIEF DESCRIPTION OF THE TABLES

Illustrated aspects of the disclosure are described in detail below with reference to attached Tables, which are incorporated by reference herein and where:

Table 1 provides statistical analysis of nutrient digestion, dry matter intake (DMI), growth, average daily gain (ADG), feed efficiency, and cost of gain.

Table 2 provides statistical analysis of nutrient digestion, DMI, growth, ADG, feed efficiency, and cost of gain.

Table 3 provides statistical analysis of blood serum chemistry analysis including blood urea nitrogen (BUN) and total dissolved carbon dioxide (TCO₂).

Table 4 provides statistical analysis of dietary treatment nutrition analysis including dry matter (DM), crude protein (CP), acid detergent fiber (ADF), neutral detergent fiber (NDF), and water-soluble carbohydrates (WSC), net energy of gain (NEG), and net energy of maintenance (NEM).

### DETAILED DESCRIPTION

Increasing metabolizable energy and protein intake has been shown to be positively correlated to both body growth and feed efficiency metrics in heifer calves. With increased metabolizable energy supply, positive correlations between blood serum glucose and blood serum insulin growth factor I (IGF-1) and skeletal development (Brown et al., 2005). Increased development has been shown to be directly related to blood serum glucose, IGF-1 concentrations, metabolizable energy intake and growth and development (Kerr et al., 1991; Nosbush et al., 1996). Improving the metabolizable energy intake through diet manipulation postweaning has multiple economic benefits including improved growth, mammary development, ovarian follicle count, and lifetime production (Diaz et al., 2001; Bach, 2011; Geiger et al., 2016; Bruinjé et al., 2019). It therefore follows in order to stimulate skeletal growth and development postweaning, increasing metabolizable energy intake and blood serum glucose levels are of primary importance.

During the germination of small grains, free glucose and amino acid levels greatly increase. The hydroponically sprouted cereal grains process primarily benefits from the hydrolysis of complex storage compounds in the seed into simpler highly bioavailable nutrient forms within the harvested product. Leveraging metabolic processes common to higher plants during germination and seedling development, hydroponically sprouted cereal enables the transformation of complex starches, proteins, and lipids into their simpler monosaccharide, amino acid, and fatty acid precursors, respectively. In a six-day period, approximately 75% of the starting starch concentration and 90% of the starting protein concentration is hydrolyzed into simpler precursors within the hydroponically sprouted cereal grains (Koehler et al., 2007).

As a novel approach to improve growth including skeletal development in dairy cattle, hydroponically sprouted cereal grains may be used as a sustainable and scalable solution to stimulate metabolizable energy intake postweaning. Furthermore, through increasing relative concentration of hexoses and in turn propionate, gluconeogenesis, and insulin levels, hydroponically sprouted cereal grains may be used as an approach to positively impact not only energy balance, but blood serum glucose and IGF-1 concentrations.

In one aspect of the present disclosure, a novel approach using a soilless seeding system to grow soilless sprouted cereal grains may be used improve skeletal development within ruminants. The soilless seeding system may include systems utilizing hydroponics, aeroponics, aquaponics, semi-hydroponics, passive hydroponics, or any method for growing plants without soil. The soilless seeding system may provide all nutrients to the plant through a nutrient solution that meets all the plant's requirements. The soilless seeding system may use a soilless growth medium. The soilless growth medium may include air, water, inert matter, or inorganic matter such as clay balls, lightweight expanded clay aggregate, pumice, loose gravel, river rock, sand, foam, rock wool, vermiculite, perlite or any other soilless mixes. Increasing relative concentration of hexoses thereby influences rates of glycolysis through inclusion of soilless sprouted grains to theoretically increase insulin level and glucose levels. Feeding soilless sprouted grains or elevated levels of complex carbohydrates often decreases ruminal pH as a direct effect of increased acetate production. Furthermore, through increasing relative concentration of hexoses and in turn propionate, gluconeogenesis, and insulin levels, hydroponically sprouted cereal grains may be used as an approach to positively impact not only energy balance, but blood serum glucose and IGF-1 concentrations.

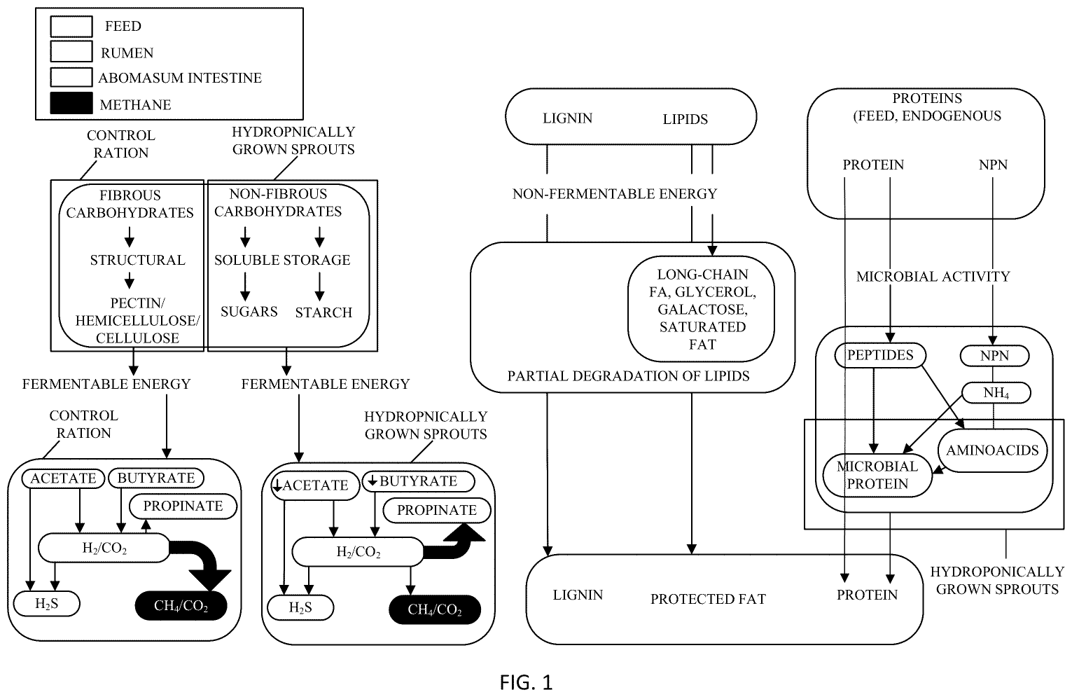

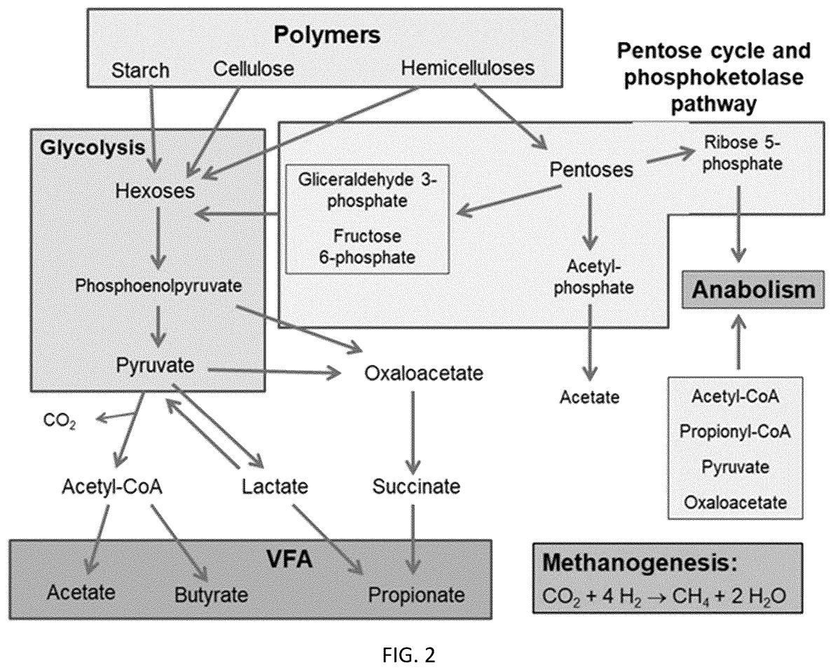

The processes, systems, and methods of the present disclosure improve skeletal development in prepubescent ruminants. The process, systems, and method of the present disclosure provide hydroponically sprouted grains or hydroponically sprouted cereal grains as a sustainable and scalable solution to improve skeletal development and nutritional management in ruminants post weaning. The processes and methods of the present disclosure increase relative concentration of hexoses and thereby rates of glycolysis through inclusion of hydroponically sprouted grains to reduce dihydrogen concentrations within the rumen. Through increasing relative concentration of hexoses and in turn propionate, gluconeogenesis, and insulin levels, hydroponically sprouted cereal grains may be used as an approach to positively impact not only energy balance, but weight, height, and length of a ruminant. Favoring glycolysis through increased supplementation of hexoses supports increased production of propionate through lactate lowering acetyl-CoA and carbon dioxide pathways, as shown in FIG. 1 and FIG. 2. Especially relevant to skeletal development, elevated levels of propionate in comparison to acetate favor increased insulin growth factor 1 (IGF-1) levels through the strong connection between plasma insulin and blood serum glucose concentration and propionate production (Fluharty 2003; Bines et al., 1984, Oh et al., 2015). Hydrolysis reactions external to the rumen, e.g. enzymatic hydrolysis of hemicelluloses during germination; improve efficiency of energy utilization, reduce hydrogen dioxide and carbon dioxide formation within the rumen improving feed efficiency, lower the acetate to propionate volatile fatty acid ratio supporting positive progesterone profile shifts, improve gluconeogenesis supporting improved energy balance, and support skeletal development within the animal.

Endogenous enzymes produced during controlled hydroponic germination of seeds can be used for enhancing the nutrient digestion capabilities of animal feedstuffs including feed concentrates, forages, and mineral supplements. Leveraging metabolic processes common to higher plants during germination and seedling development, the grower system enables the transformation of complex polysaccharides including starch and cellulose, complex proteins, and triglycerides into their reduced monosaccharide, amino acid, and fatty acid precursors, respectively. When enzymes are incorporated into a high starch diet, such as by increasing the amount of enzymes within a plant and allowed time to act before animal digestion there is an overall impact on nutrient digestibility. In a six-day period, approximately 75% of the starting starch concentration and 90% of the starting protein concentration may be hydrolyzed into simpler precursors within developing cereal grains. Thereby improving efficiency of energy utilization in the ruminant, reducing hydrogen dioxide and carbon dioxide formation within the rumen improving feed efficiency, lowering the acetate to propionate volatile fatty acid ratio supporting positive progesterone profile shifts, improving gluconeogenesis supporting improved energy balance, and supporting the development of reproductive structures within the animal.

The plant or seed may refer to any plant from the kingdom Plantae or angiosperms including flowering plants, cereal grains, grain legumes, grasses, roots and tuber crops, vegetable crops, fruit plants, pulses, medicinal crops, aromatic crops, beverage plants, sugars and starches, spices, oil plants, fiber crops, latex crops, food crops, feed crops, plantation crops or forage crops.

Cereal grains may include rice (Oryza sativa), wheat (Triticum), maize (Zea mays), rye (Secale cereale), oat (Avena sativa), barley, (Hordeum vulgare), sorghum (Sorghum bicolor), pearl millet (Pennisetum glaucum), finger millet (Eleusine coracana), barnyard millet (Echinochloa frumentacea), Italian millet (Setaria italica), kodo millet (Paspalum scrobiculatum), common millet (Panicum miliaceum).

Pulses may include black gram, kalai, or urd (Vigna mungo var, radiatus), chickling vetch (Lathyrus sativus), chickpea (Cicer arietinum), cowpea (Vigna sinensis), green gram mung (Vigna radiatus), horse gram (Macrotyloma uniflorum), lentil (Lens esculenta), moth bean (Phaseolus aconitifolia), peas (Pisum sativum) pigeon pea (Cajanus cajan, Cajanus indicus), philipesara (Phaseolus trilobus), soybean (Glycine max).

Oilseeds may include black mustard (Brassica nigra), castor (Ricinus communis), coconut (Cocus nucifera), peanut (Arachis hypogea), Indian mustard (Brassica juncea), toria (Napus), niger (Guizotia abyssinica), linseed (Linum usitatissimum), safflower (Carthamus tinctorius), sesame (Sesamum indicum), sunflower (Helianthus annus), white mustard (Brassica alba), oil palm (Elaeis guineensis). Fiber crops may include sun hemp (Crotalaria juncea), jute (Corchorus), cotton (Gossypium), mesta (Hibiscus), or tobacco (Nicotiana).

Sugar and starch crops may include potato (Solanum tuberosum), sweet potato (Ipomea batatus), tapioca (Manihot esculenta), sugarcane (Saccharum officinarum), sugar beet (Beta vulgaris). Spices may include black pepper (Piper nigrum) betel vine (Piper betle), cardamom (Elettaria cardamomum), garlic (Allium sativum), ginger (Zingiber officinale), onion (Allium cepa), red pepper or chillies (Capsicum annum), or turmeric (Curcuma longa). Forage grasses may include buffel grass or anj an (Cenchrus ciliaris), dallis grass (Paspalum dilatatum), dinanath grass (Pennisetum), guniea grass (Panicum maximum), marvel grass (Dicanthium annulatum), napier or elephant grass (Pennisetum purpureum), pangola grass (Digitaria decumbens), para grass (Brachiaria mutica), sudan grass (Sorghum sudanense), teosinte (Echlaena mexicana), or blue panicum (Panicum antidotale). Forage legume crops may include berseem or Egyptian clover (Trifolium alexandrinum), centrosema (Centrosema pubescens), gaur or cluster bean (Cyamopsis tetragonoloba), Alfalfa or lucerne (Medicago sativa), sirato (Macroptlium atropurpureum), velvet bean (Mucuna cochinchinensis).

Plantation crops may include banana (Musa paradisiaca), areca palm (Areca catechu), arrowroot (Maranta arundinacea), cacao (Theobroma cacao), coconut (Cocos nucifera), Coffee (Coffea arabica), tea (Camellia theasinesis). Vegetable crops may include ash gourd (Beniacasa cerifera), bitter gourd (Momordica charantia), bottle gourd (Lagenaria leucantha), brinjal (Solanum melongena), broad bean (Vicia faba), cabbage (Brassica), carrot (Daucus carota), cauliflower (Brassica), colocasia (Colocasia esulenta), cucumber (Cucumis sativus), double bean (Phaseolus lunatus), elephant ear or edible Arum (Colocasia antiquorum), elephant foot or yam (Amorphophallus campanulatus), French bean (Phaseolus vulgaris), knol khol (Brassica), yam (Dioscorea) lettuce (Lactuca sativa), musk melon (Cucumis melo), pointed gourd or parwal (Trichosanthes diora), pumpkin (Cucrbita), radish (Raphanus sativus), bhendi (Abelmoschus esculentus), ridge gourd (Luffa acutangular), spinach (Spinacia oleracea), snake gourd (Trichosanthes anguina), tomato (Lycoperscium esculentus), turnip (Brassica), or watermelon (Citrullus vulgaris).

Medicinal crops may include aloe (Aloe vera), ashwagnatha (Withania somnifera), belladonna (Atropa belladonna), bishop's weed (Ammi visnaga), bringaraj (Eclipta alba.), cinchona (Cinchona sp.) coleus (Coleus forskholli), dioscorea, (Dioscorea), glory lily (Gloriosa superba), ipecae (Cephaelis ipecauanha), long pepper (Poper longum), prim rose (Oenothera lamarekiana), roselle (Hibiscus sabdariffa), sarpagandha (Rauvalfia serpentine) senna (Cassia angustifolia), sweet flag (Acorus calamus), or valeriana (Valeriana wallaichii).

Aromatic crops may include ambrette (Abelmoschus moschatus), celery (Apium graveolens), citronella (Cymbopogon winterianus), geranium (Pelargonium graveolens.), Jasmine (Jasminum grantiflorum), khus (Vetiveria zizanoids), lavender (Lavendula sp.) lemon grass (Cymbopogon flexuosus), mint, palmarosa (Cymbopogon martini), patchouli (Pogostemon cablin), sandal wood (Santalum album), sacred basil (Ocimum sanctum), or Tuberose (Polianthus tuberosa). Food crops are harvested for human consumption and feed crops are harvested for livestock consumption. Forage crops may include crops that animals feed on directly or that may be cut and fed to livestock.

Nutrient digestibility is the amount of nutrients absorbed by the individual or animal and is generally calculated as the amount of nutrients consumed minus the amount of nutrients retained in the feces. The incorporation of enzymes into dairy and beef rations has yielded mixed results and has primarily been focused on amylase in cattle. The incorporation of amylase into dairy and beef rations has been shown to increase milk to feed conversions by twelve percent when 15,000 kilo novo units (KNUs) were supplied in a starch rich ration. In beef cattle, the addition of 12,000 KNUs of exogenous amylase improved the daily rate of gain by eight percent. The direct influence of amylase of milk yield and components is mixed with increases in milk and milk components reported by a few authors. Consistently across trials the addition of amylase has been reported to improve nutrient digestibility and feed use efficiency. The use of enzymes produced during the germination process of cereal grains has long been used in application for the malting industry, the process of leveraging enzymes produced during the optimized hydroponic germination of seeds has yet to be implemented in the feed industry to improve the skeletal development, nutritional management, metabolic energy, and milk production within the animal.

The processes, systems, and methods of the present disclosure improve skeletal development in ruminants. The processes, systems, and methods of the present disclosure provide hydroponically sprouted grains or hydroponically sprouted cereal grains as a sustainable and scalable solution to improve skeletal development and metabolizable energy in prepubescent ruminants. The processes and methods of the present disclosure increase relative concentration of hexoses and thereby rates of glycolysis through inclusion of hydroponically sprouted grains to reduce dihydrogen concentrations within the rumen. Through increasing relative concentration of hexoses and in turn propionate, gluconeogenesis, and insulin levels, hydroponically sprouted cereal grains may be used as an approach to positively impact not only energy balance, but skeletal development and growth. Favoring glycolysis through increased supplementation of hexoses supports increased production of propionate through lactate lowering acetyl-CoA and carbon dioxide pathways.

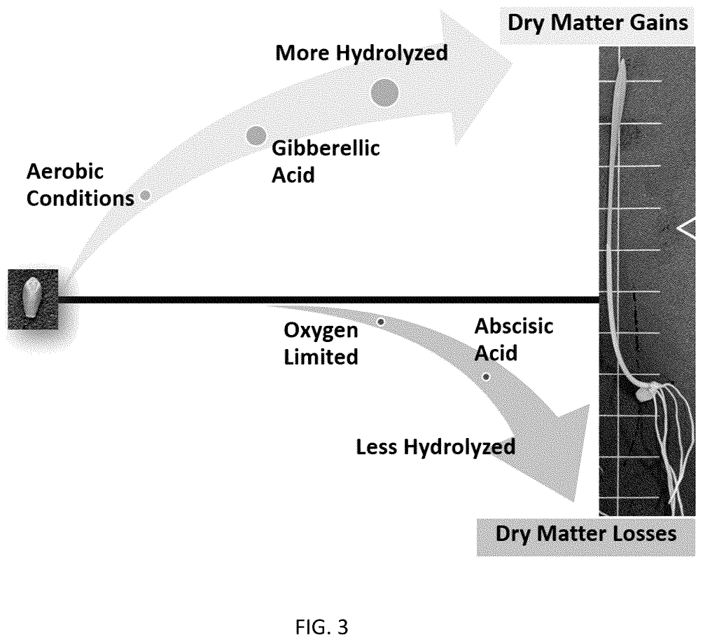

Poor quality forages, such as hay and silages, can be responsible for inferior growth performance in prepubescent heifers. Both of these forages typically have low protein, mineral, and vitamin levels. A lack of a balanced grain supplement may exacerbate or add to the problem. Underfed or malnourished calves typically do not receive adequate energy and protein to meet their growth requirements. Adequate weight but restricted skeletal growth may occur in prepubescent heifers by relying on poor quality hay and corn silage for all or most of the heifers' diet. Therefore, what is need is a process, method and system for nutritional management and improving skeletal development in prepubescent ruminants by utilizing hydroponically sprouted cereal grains. Plant growth and the production of enzymes are greatly affected by the environment. Most plant problems, such as decreased nutrient digestibility, are caused by environmental stresses due to environmental conditions. Environmental factors such as water, humidity, nutrition, light, temperature, and level of oxygen present can affect a plant's growth and development as shown in FIGS. 3-5.

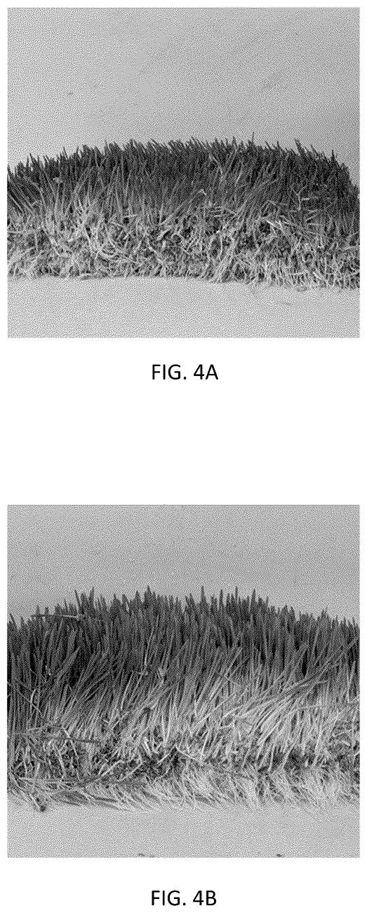

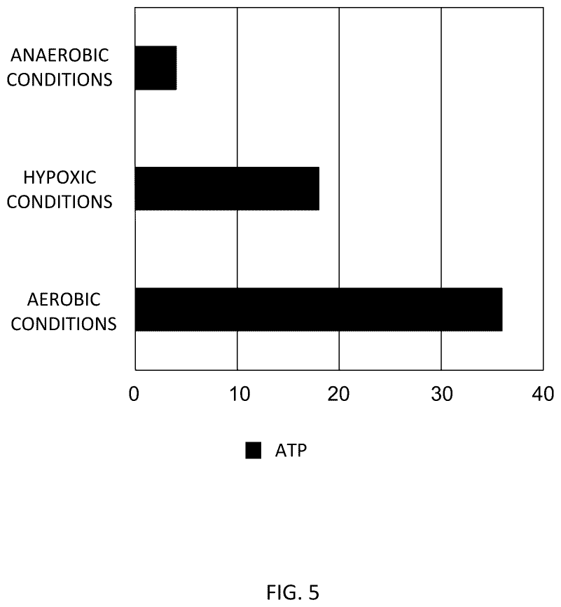

Oxygen is a necessary component in many plant processes included respiration and nutrient movement from the soil into the roots. The amount of oxygen can influence the efficiency of respiration. Oxygen moves passively into the plant through diffusion. Plants may be affected by growing in anaerobic conditions, where the uptake or disappearance of oxygen is greater than its production by photosynthesis or diffusion by physical transport from the surrounding environment. Anaerobic conditions can cause nutrient deficiencies or toxicities within the plant, root or plant death, or reduced growth of the plant. Anaerobic conditions may be caused by a decrease in the amount of oxygen in the air, such as growing a plant or seed in a room without air or oxygen circulation. However, oxygen bound in compounds such as nitrate (NO₃), nitrite (NO₂), and sulfites (SO₃) may still be present in the environment. Waterlogging, where excess water is present in the root zone of the plant or in the soil, inhibits gaseous exchange with the air and can also cause anaerobic conditions. Hypoxic conditions arise when there is insufficient oxygen in a plant's environment and the plant must adapt its growth and metabolism accordingly. Excessive watering or waterlogged soil can cause hypoxic conditions. When anaerobic or hypoxic conditions persist, the microbial, fungal and plant activities quickly use up any remaining oxygen. The plant becomes stressed due to the lack of nutrient uptake by the roots, the plant stomata begin to close, and photosynthesis is reduced. A prolonged period of oxygen deficiency can lead to reduced yields, root dieback, plant death, or greater susceptibility to disease and pests as shown in FIG. 4A. Under aerobic conditions plant growth can thrive, as shown in FIG. 4B. Aerobic conditions are when there is enough oxygen molecules or compounds and energy present to carry out oxidative reactions including nutrient cycling, as shown in FIG. 5.

Light is a necessary component for plant growth and the increase in the production of enzymes, sugars and starches that increase nutrient digestibility. The more light a plant receives, the greater its capacity for producing food and energy via photosynthesis. The energy can be used to produce or increase the expression of enzymes that increase nutrient digestibility. Temperature influences most plant processes, including photosynthesis, transpiration, respiration, germination, and flowering. As temperature increases up to a certain point, photosynthesis, transpiration, and respiration increase. When the temperature is too low or exceeds the maximum point photosynthesis, transpiration, and respiration decrease. When combined with day-length, temperature also affects the change from vegetative to reproductive growth. The temperature for germination may vary by plant species. Generally, cool-season crops (e.g., spinach, radish, and lettuce) germinate between 55° to 65° F., while warm-season crops (e.g., tomato, petunia, and lobelia) germinate between at 65° to 75° F. Low temperatures reduce energy use and increase simple sugar storage whereas adverse temperatures, however, cause stunted growth and poor-quality plants.

Water and humidity play an important role in increasing nutrient digestibility. Most growing plants contain ninety percent water, Water is the primary component of photosynthesis and respiration. Water is also responsible for the turgor pressure needed to maintain cell shape and ensure cell growth. Water acts as a solvent for minerals and carbohydrates moving through the plant, acts as a medium for some plant biochemical reactions, increases enzyme production and expression, and cools the plant as it evaporates during transpiration. Water can regulate stomatal opening and closing thereby controlling transpiration and photosynthesis and is a source of pressure for moving roots through a growing medium such as soil. Humidity is the ratio of water vapor in the air to the amount of water the air can hold at the current temperature and pressure. Warm air can hold more water vapor than cold air. Water vapor moves from an area of high humidity to an area of low humidity. Water vapor moves faster if there is a greater difference between the area of high humidity and the area of low humidity. When the plant's stoma open, water vapor rushes outside the plant into the surrounding air. An area of high humidity forms around the stoma and reduces the difference in humidity between the air spaces inside the plant and the air adjacent to the plant, slowing down transpiration. If air blows the area of high humidity around the plant away, transpiration increases.

Plant nutrition plays an important role in increasing nutrient digestibility. Plant nutrition is the plant's need for and use of basic chemical elements. Plants need at least 17 chemical elements for normal growth. Carbon, hydrogen, and oxygen can be found in the air or in water. The macronutrients, nitrogen, potassium, magnesium, calcium, phosphorus, and sulfur are used in relatively large amounts by plants. Nitrogen plays a fundamental role in energy metabolism, protein synthesis, and is directly related to plant growth. It is indispensable for photosynthesis activity and chlorophyll formation. It promotes cellular multiplication. A nitrogen deficiency results in a loss of vigor and color. Growth becomes slow and leaves fall off, starting at the bottom of the plant. Calcium attaches to the walls of plant tissues, stabilizing the cell wall and favoring cell wall formation. Calcium aids in cell growth, cell development and improves plant vigor by activating the formation of roots and their growth. Calcium stabilizes and regulates several different processes. Magnesium is essential for photosynthesis and promotes the absorption and transportation of phosphorus. It contributes to the storage of sugars within the plant. Magnesium performs the function of an enzyme activator. Sulfur is necessary for performing photosynthesis and intervenes in protein synthesis and tissue formation.

The plant micronutrients or trace elements, iron, zinc, molybdenum, manganese, boron, copper, cobalt, and chlorine, are used by the plant in smaller amounts. Macronutrients and micronutrients can be dissolved by water and then absorbed by a plant's roots. A shortage in any of them leads to deficiencies, with different adverse effects on the plant's general state, depending upon which nutrient is missing and to what degree. Fertilization may affect nutrient digestibility. Fertilization is when nutrients are added to the environment around a plant. Fertilizers can be added to the water or a plant's growing surface, such as soil. Additional micronutrients and macronutrients can be added to the plant by the grower system.

Plant growth can be split into four growing stages: imbibition, plateau, germination, and seedling. Imbibition is the uptake of water by a dry seed. As the seed intakes the water, the seed expands, enzymes and food supplies become hydrated. The enzymes become active, and the seed increases its metabolic activity. During imbibition the relative humidity is high and may range from 90% to 98% relative humidity. The temperature may range from 76° F. to 82° F. or 22° C. to 28° C. Air movement is minimal. The imbibition may last 18 to 24 hours. The plateau stage is where water uptake increases very little. The plateau stage is associated with hormone metabolism such as abscisic acid (ABA) and gibberellic acid (GA) synthesis or deactivation. During the plateau stage humidity and temperature may be lower than the imbibition stage. Relative humidity may range from 70% to 90% and the temperature may range from 72° F. to 77° F. or 22° C. to 26° C. Air movement may still be minimal. The plateau stage may last 18-24 hours. Germination is the sprouting of a seed, spore, or other reproductive body. The absorption of water, temperature, oxygen availability and light exposure may operate in initiating the process. During germination, the relative humidity may be lower than the imbibition and plateau stage. Relative humidity may range from 60% to 70%. The temperature may be the same as the plateau stage and range from 72° F. to 77° F. or 22° C. to 26° C. Air movement may be moderate. Germination may last 24 to 48 hours. The last phase is the seedling or plant development phase where the plant's roots develop and spread, nutrients are absorbed fueling the plants rapid growth. The seedling stage may last until the plant matures. The seedling stage may also be broken down into additional phases: seedling, budding, flowering and ripening. The relative humidity may be lowest at this stage and range from 40% to 60%. The temperature may also be the lowest at this stage and range from 68° F. to 72° F. or 20° C. to 22° C. Air movement is high. The seedling phase can range from 72 hours or until the plant reaches maturity. The specific control of temperature encourages maximum enzyme hydrolysis throughout development while potentially discouraging the cellular division near the onset of photosynthesis. Temperatures near the cardinal range of seeds is believed to support maximum enzyme hydrolysis approximately through the first 120 hours. Reducing temperatures below the cardinal value at 120 hours is believed to reduce metabolic activity in tissue readily exposed to the environment while having reduced influence on the seed within the cellulosic material layer.

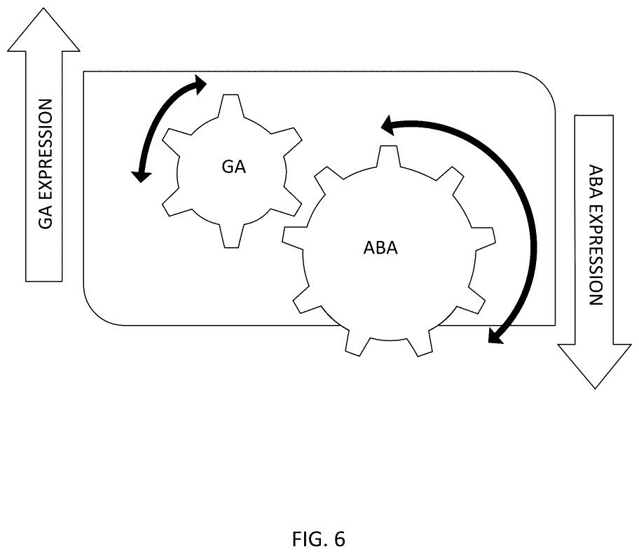

Phytohormones, such as abscisic acid (ABA), GA and ethylene (ET) regulate seed dormancy and seed germination as well as balance or dictate enzyme production. The ratio of ABA and GA regulates seed dormancy. When levels of ABA are high, stomatal closure, stress signaling and delay in cell division is triggered down regulating metabolic and enzyme activity. High ABA/GA ratios favor dormancy, whereas low ABA/GA ratios result in seed germination. The increase in GA is necessary for seed germination to occur, as GA expression increases, ABA expression decreases, as shown in FIG. 6. GA triggers cell division, stem elongation and root development. Enzyme expression is closely linked to metabolic needs during germination. As the plant becomes metabolically active shortly after imbibition, GA is released from the seed embryo signaling the release of a wide profile of enzymes from within the seed including from the aleurone layer surrounding the polysaccharide and protein rich endosperm of the seed. During germination, GA translocates to and interacts with the aleurone layer, thereby releasing or synthesizing hydrolytic enzymes, included α-amylase. The term “amylase” means an enzyme that hydrolyzes 1,4-alpha-glucosidic linkages in oligosaccharides and polysaccharides, including the following classes of enzymes: alpha-amylase, beta-amylase, glucoamylase, and alpha-glucosidase.

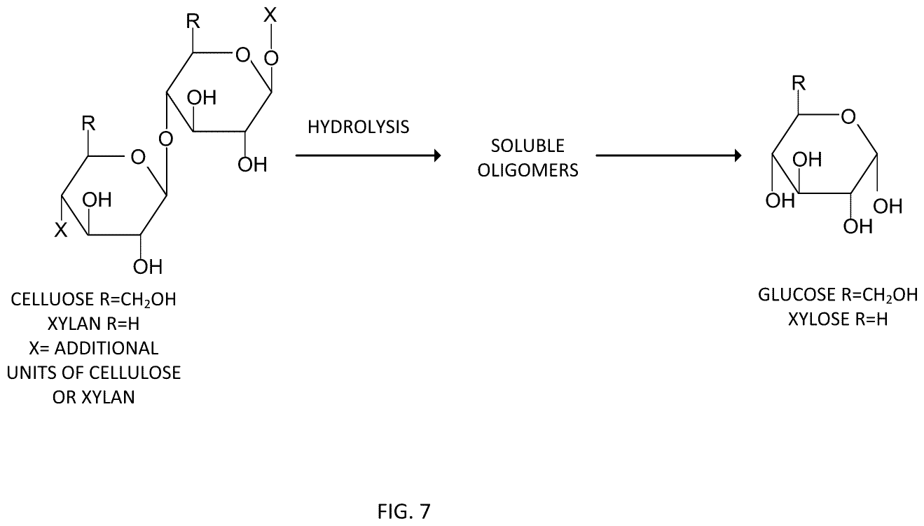

Hydrolytic enzymes are some of the most energy efficient enzymes. The hydrolytic enzymes, such as 1,3;1,4-β-glucanase (β-glucanase), α-amylase and β-amylase, are released. The term “beta-glucosidase” means a beta-D-glucoside glucohydrolase that catalyzes the hydrolysis of terminal non-reducing beta D-glucose residues with the release of beta-D-glucose. Once the hydrolytic enzymes are released, they facilitate the hydrolysis of complex storage molecules including cell wall polysaccharides, proteases, storage proteins, and starchy energy reserves that are essential for germination, providing sugars that drive the root growth, into their simpler monomer subunits. Hydrolysis of the storage molecules is one of the primary energy sources of plants. Hydrolysis of storage molecules into their simpler monomer subunits, increases the rate at which the ruminant can digest the feed and utilize the feed for energy, thereby increasing feed efficiency. The hydrolytic enzymes break the polymers into dimers or soluble oligomers and then into monomers by water splitting the chemical bonds, as shown in FIG. 7.

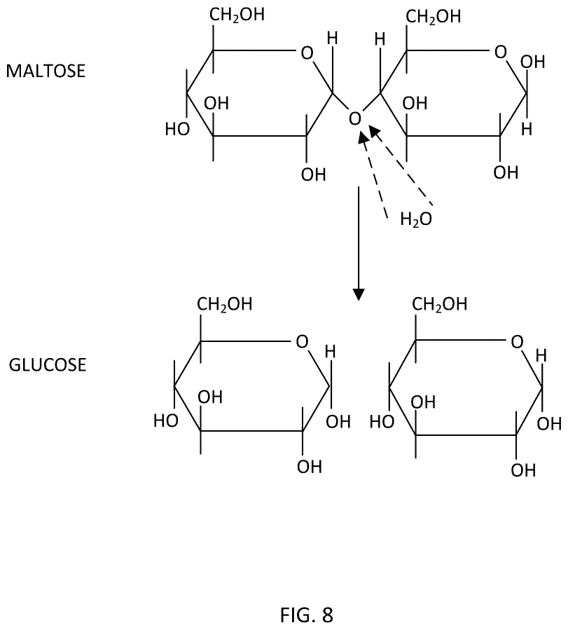

3-glucanase may hydrolyze 1,3;1,4-β-glucan, a predominant cell wall polysaccharide. The α-amylase cleaves internal amylose and amylopectin residues. The β-amylase exo-hydrolase liberates maltose and glucose from the starch molecules as shown in FIG. 8. These reduced nutrient forms are commonly then transported back to the embryo where glycolysis and the cellular respiration pathway uses glucose to produce ATP needed for energy intensive cellular division and biosynthesis reactions. As the metabolic needs of the juvenile plant increases, the release of GA from the seed embryo and the release of enzymes from the aleurone layer likewise increases. Enzyme activity within the juvenile plant peaks at the onset of efficient photosynthesis. At this point, the total metabolic demands of the plant are not able to be met by photosynthesis and a large amount of storage molecules must be hydrolyzed to glucose for glycolysis and ATP generation.

Most mammals have a hard time digesting dietary fibers including cellulose. Cellulose polysaccharides are the prominent biomass of the primary cell wall, followed by hemicellulose and pectin. Cellulosic material is any material containing cellulose. The secondary cell wall, produced after the cell has stopped growing, also contains polysaccharides and is strengthened by polymeric lignin covalently cross-linked to hemicellulose. Cellulose is a homopolymer of anhydrocellobiose and is a linear beta-(1-4)-D-glucan. Hemicellulose can include a variety of compounds, such as, Xylans, Xyloglucans, arabinoxylans, and mannans in complex branched structures with a spectrum of Substituents. Cellulose, although polymorphous, is primarily found as an insoluble crystalline matrix of parallel glucan chains. Hemicellulose usually hydrogen bonds to cellulose as well as other hemicelluloses, stabilizing the cell wall matrix. Cellulolytic enzymes or cellulase mean one or more enzymes that hydrolyze a cellulosic material. The enzymes may include endoglucanase(s), cellobiohydrolase(s), beta-glucosidase(s), or combinations thereof. The enzymes break the cellulosic material down into cellodextrins or completely into glucose. Hemmicellulolytic enzyme or hemicullase are one or more enzymes that hydrolyze a hemicellulosic material forming furfural or arabinose and xylose. When a ruminant digests the feed with the hydrolyzed hemicellulosis material, energy utilization is more efficient, hydrogen dioxide formation is reduced increasing feed efficiency, and gluconeogenesis rates are improved thereby improving energy balance and the development of reproductive structures.

Beta-xylosidase, or beta-D-xyloside xylohydrolase, catalyzes the exo-hydrolysis of short beta (1->4)-xylooligosaccharides to remove successive d-xylose residues from non-reducing termini and may hydrolyze xylobiose. Beta-xylosidase engage in the final breakdown of hemicelluloses. The term “xylanase” means a 1,4-beta D-xylan-Xylohydrolase that catalyzes the endohydrolysis of 1,4-beta-D-Xylosidic linkages in Xylans. The term “endoglucanase” means an endo-1,4-(1,3:1,4)-beta-D-glucan 4-glucanohydrolase that catalyzes endohydrolysis of 1,4-beta-Dglycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or Xyloglucans, and other plant material containing cellulosic components.

Lignin is another primary component of the cell wall. Lignin is a class of complex polymers that form key structural materials in support tissues, such as the primary cell wall, in most plants. The lignols that crosslink to form lignins are of three main types, all derived from phenylpropane: coniferyl alcohol (4-hydroxy-3-methoxyphenylpropane), sinapyl alcohol (3,5-dimethoxy-4-hydroxyphenylpropane), and paracoumaryl alcohol (4-hydroxyphenylpropane. Lignin fills the spaces in the cell wall between cellulose, hemicellulose, and pectin components. It can covalently crosslink to hemicellulose mechanically strengthening the cell wall. Ligninolytic enzymes are enzymes that hydrolyze lignin polymers. The ligninolytic enzymes include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic Sugars (notably arabinose) and lignin.

Lipids are used as structural components to limit water loss and pathogen infection. These lipids include waxes derived from fatty acids, as well as cutin and Suberin. Lipase is an enzyme that hydrolyzes lipids, fatty acids, and acylglycerides, including phosphoglycerides, lipoproteins, diacylglycerols, and the like. Lipases include the following classes of enzymes: triacylglycerol lipase, phospholipase A2, lysophospholipase, acylglycerol lipase, galactolipase, phospholipase A1, dihydrocoumarin lipase, 2-acetyl-1-alkylglycerophosphocholine esterase, phosphatidylinositol deacylase, cutinase, phospholipase C, phospholipase D, 1-hosphatidylinositol phosphodiesterase, and alkylglycerophospho ethanolamine phosphdiesterase. Lipase increases the digestibility of lipids by breaking lipids down digestibly into saccharides, disaccharides, and monomers.

Phytate is the main storage form of phosphorous in plants. However, many animals have trouble digesting or are unable to digest enzymes because they lack enzymes that break phytate down. Because phosphorus is an essential element, inorganic phosphorous is usually added to animal feed. Phytase is a hydrolytic enzyme that specifically acts on phytate, breaking it down and releasing organic phosphorous. The term “phytase” means an enzyme that hydrolyzes ester bonds within myo-inositol-hexakisphosphate or phytin, including 4-phytase, 3-phytase, and 5-phytase. By increasing the activity of the hydrolytic enzymes, organic phosphorous is released and inorganic phosphorous does not have to be added to animal feed.

Protease breaks down proteins and other moieties, such as sugars, into smaller polypeptides and single amino acids by hydrolyzing the peptide bonds. Many of the proteins serve as storage proteins. Some specific types of proteases include cysteine proteases including pepsin, papain and serine proteases including chymotrypsins, carboxypeptidases and metalloen dopeptidases. Proteases play a key role in germinations through the hydrolysis and mobilization of proteins that have accumulated in the seed. Proteases also play a role in programmed cell death, senescence, abscission, fruit ripening, plant growth, and N homeostasis. In response to abiotic and biotic stresses, proteases are involved in nutrient remobilization of leaf and root protein degradation to improve yield.

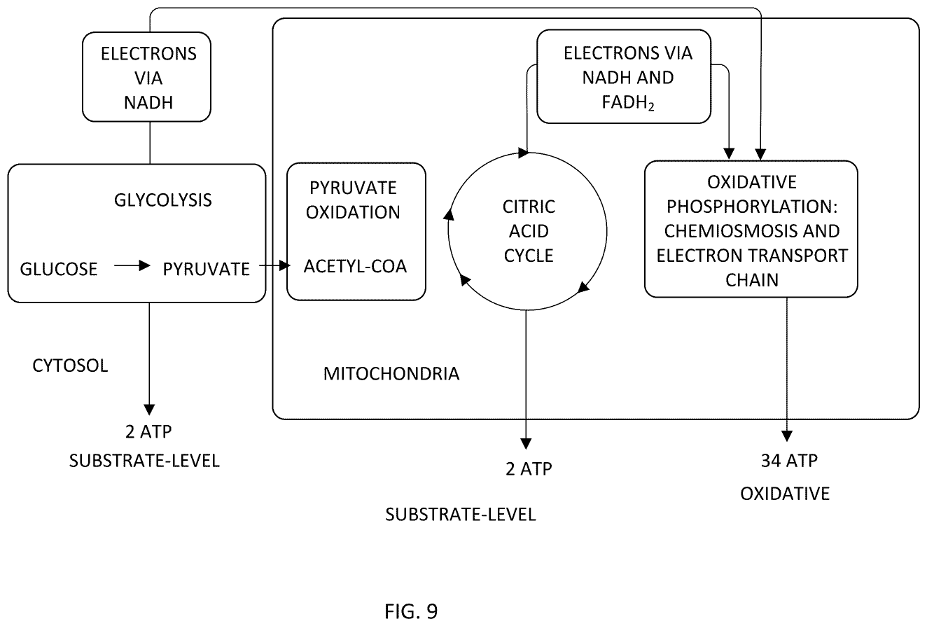

Cellular respiration is a set of metabolic reactions that take place in the cells of the seed to convert chemical energy from oxygen molecules or nutrients into adenosine triphosphate (ATP), as shown in FIG. 9. Nutrients, such as sugar, amino acids and fatty acids are used during cellular respiration. Oxygen is the most common oxidizing agent. Aerobic respiration requires oxygen to create ATP and is the preferred method of pyruvate in the breakdown into glycolysis. The energy transferred is used to break bonds in adenosine diphosphate (ADP) to add a third phosphate group to form ATP by phosphorylation, nicotinamide adenine dinucleotide (NADH) and flavin adenine dinucleotide (FADH₂). NADH and FADH₂ is converted to ATP using the electron transport chain with oxygen and hydrogen being the terminal electron acceptors. Most of the ATP produced during aerobic cellular respiration is made by oxidative phosphorylation. Oxygen releases chemical energy which pumps protons across a membrane creating a chemiosmotic potential to drive ATP synthase.

By decreasing environmental stresses and increasing metabolic activity, the plant can be harvested in an interval that closely aligns with the maximum point of enzyme activity within the plant's life cycle and increased development results. The harvested product is rich in enzymes. Based on enzyme values reported when investigating the malting characteristics of cereals, barley is estimated to have approximately 12,000 kilo novo units (KNO) of amylase activity per kg dry matter, 400 units of protease per milligram protein and 200 units of lipase per milligram protein. Wheat is expected to have amylase levels approximately 50% to 75% the amount of barley on average with lipase and protease values equal and 100% greater, respectively. Enzymes, such as peroxidase and hemicellulose, relating to fiber catabolism are likely also very active due to the decrease in environmental stresses.

For example, barley harvested at the maximum point of enzyme activity, the amount of crude protein increases. Crude protein is the content of the animal feed or plant sample that represents the total nitrogen, including true protein and non-protein nitrogen (urea and ammonia). Crude protein is an important indicator of the protein content of a forage crop. In one example the crude protein in barley can be increased by 143% instead of 117% and 125% when harvested on day six, when enzyme activity was maximized. In another example, wheat is harvested at the maximum enzyme point, such as day six, the amount of crude protein can be increased by 129%. The neutral detergent fiber (NDF) of a crop, plant, or feed sample content is a close estimate of the total fiber constituents of the crop. The NDF contains plant cell wall components such as cellulose, hemicellulose, lignin, silica, tannins, and cutins, it does not include some pectins. The structural carbohydrates, hemicellulose, cellulose, and lignin, represent the fibrous bulk of the crop. Though lignin is indigestible, hemicellulose and cellulose can be (in varying degrees) digested by microorganisms in animals with either a rumen, such as cattle, goats or sheep, or hind-gut fermentation such as horses, rabbits, guinea pigs, as part of their digestive tract. NDF is considered to be negatively correlated with dry matter intake, as when the percentage of NDF increases the animals consume less of the crop. In one example the NDF in barley can be increased by 178% instead of by 132% and 155% when harvested on day six when enzyme activity is maximized. In another example, when wheat is harvested at the maximum enzyme point, such as day six, the amount of NDF can be increased by 173%. Water-soluble carbohydrates (WSC) are carbohydrates that can be solubilized and extracted in water. WSC's can include monosaccharides, disaccharides and a few short chain polysaccharides, such as fructans, which are major storage carbohydrates.

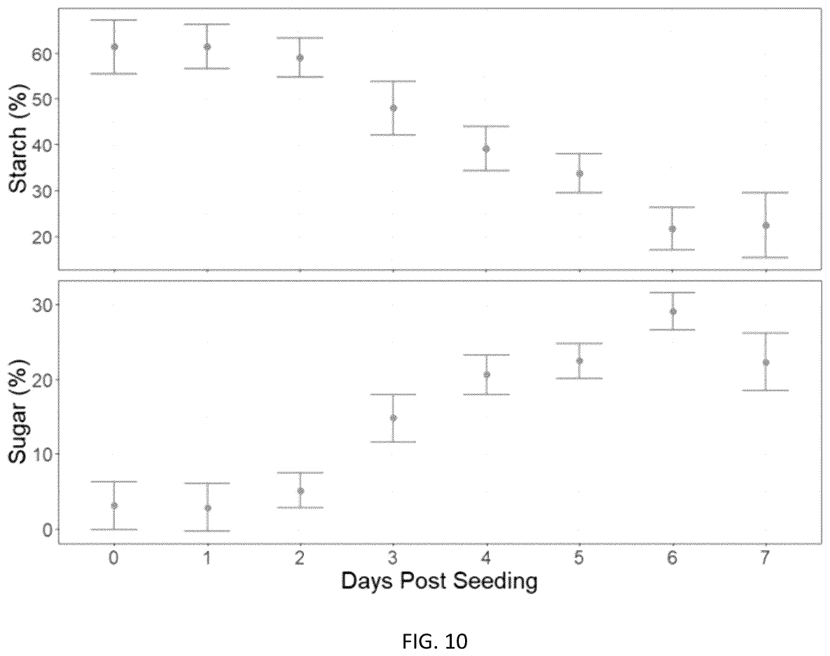

In one example the WSC in barley increased by 442% instead of by 182% and 191% when harvested on day six when enzyme activity was maximized. In another example, when wheat is harvested at the maximum enzyme point, such as day six, the amount of WSC can be increased by 553%. The increase in percentage is evidence that by increasing the enzyme activity in plants, complex storage molecules are being broken down into simpler monomer storage molecules increasing nutrient digestibility. Starch is an intracellular carbohydrate found primarily in the grain, seed, or root portions of a plant as a readily available source of energy. In crops where GA activity increases, the amount of starch present in the feed is reduced. This may be due to the breakdown of starch into simpler sugars, such as glucose and maltose, by the enzymes increasing nutrient digestibility of the feed. When enzyme activity is maximized, the amount of starch in barley can be increased by 17% and by 26% in wheat. Dry matter refers to all the plant material excluding water. The nutrient or mineral content of animal feed or plant tissues may be expressed on a dry matter basis or the proportion of the total dry matter in the material. When enzyme activity is maximized the dry matter ratio can increase, such as by 118% in barley and 115% in wheat, instead of by 92% or 95%. In yet another example, when the enzyme activity is maximized the amount of starch in the hydroponically sprouted grains may decrease in percentage, such as from 60% to 20% or 30%. By utilizing the enzymes in the hydroponically sprouted grain, starch decreases and nutrient digestible sugars increase, for example from 5% to 20% or 30% or even higher, as shown in FIG. 10.

The breakdown of storage molecules into nutrient digestible monomer subunits can be increased by leveraging GA in a hydroponic environment. When GA activity is increased in crops, the crude protein content can increase, such as from 15.9% to 20.4% in rye. When ABA activity is increased, the crude protein content decreases, for example, from 15.9% to 13.7%. Crude protein content in a crop, plant, or feed sample represents the total amount in nitrogen in the diet, including protein and non-protein nitrogen. The fibrous component of a crop, plant or feed sample content represents the least digestible fiber portion. The least digestible portion includes lignin, cellulose, silica, and insoluble forms of nitrogen. Hemicellulose is not included in the least digestible portion. Crops with a higher acid detergent fiber (ADF) have a lower digestible energy. As the ADF level increases, the digestible energy level decreases. When GA activity is increased, the ADF percentage increases, such as from 9.2% to 12.8% in rye. When ABA activity increases, the ADF percentage decreases, such as from 9.2% to 4.2%. In crops where the GA activity increases the percentage of NDF increases, such as from 21.6% to 27.1% in lye. In crops, where ABA activity increases, the NDF percentage decreases, such as from 21.6% to 15.2% in rye. The ethanol soluble carbohydrates (ESC) of a plant include monosaccharides, such as glucose and fructose, and disaccharides. When GA activity increases the ESC percentage decreases slightly, as energy is needed to grow the plant or crops. In rye the ESC percentage may decrease from 35.3% to 31.7%. In rye the starch percentage decreased from 19.1% to 9.6%. However, when ABA activity increased due to environmental stressors, the amount of starch in the rye increased from 19.1% to 42.2%. Crude fat is an estimate of the total fat content of the crop or feed sample. Crude fat contains true fat (triglycerides), alcohols, waxes, terpense, steroids, pigments, ester, aldehydes, and other lipids. In feed samples where GA activity was increased due to reducing environmental stresses, the amount of crude fat increases. In rye crops the crude fat may increase from 1.39% to 2.78%. Crude fat also increases when ABA activity increases. In rye crops the crude fat percentage may increase from 1.39 to 1.44%.

As the ruminant digests the hydroponically sprouted cereal grains, including non-fibrous carbohydrates, the fermentable energy produces the volatile fatty acids acetate, propionate, and butyrate. Acetate, propionate, and butyrate concentrations and relative proportions are dependent on the amount and composition of feed ingested. However, the amount of acetate and butyrate produced is decreased along with the ratio of acetate to propionate. As the main precursor for gluconeogenesis, propionate is involved in energy homeostasis in ruminants. The volatile fatty acids are end products of fermentation of dietary carbohydrates by the anaerobic intestinal microbiota and have multiple effects on mammalian energy metabolism. Circulating volatile fatty acids play a role in the regulation of both fatty acid and glucose metabolism. These metabolic processes are vital to the energy status of an animal and affect many downstream physiological processes.

Ruminal pH is a critical factor in the normal and stable function of the rumen because of its profound effect on microbial populations and fermentation products, and on physiological functions of the rumen, mainly motility and absorptive function. The rumen harbors diverse microorganisms including bacteria, protozoa, fungi, archaea, and viruses. They play a key role in the breakdown and utilization of feedstuff carbohydrate and protein through the process of fermentation, resulting in the production of volatile fatty acids and microbial protein. The increase in propionate increases fertility rates, postpartum energy balance, and maintenance of a healthy pregnancy. Rumen microbes may thrive on glucose, increasing efficiency of the feed composition when glucose is readily available, allowing the ruminant to process the feed composition quicker and increase propionate levels. Microbial protein yield is higher, allowing the microbes to create more protein resulting in a higher microbial biomass. By utilizing hydroponically grown sprouts, prepubescent ruminants can utilize glucose quicker, more efficient energy utilization, and create more microbial protein while maintaining a consistent pH. As the main precursor for gluconeogenesis, propionate is involved in energy homeostasis in ruminants.

As propionate increases, the amount of glucose from gluconeogenesis increases. The increase in glucose may directly increase the levels of insulin circulating and increase the levels of liver IGF-1 thereby improving skeletal development. Insulin stimulates the liver to increase or promote the expression of growth hormone receptors and release IGF-1 into circulation, regulating the ruminant's skeletal development.

Leptin is an adipocyte-derived hormone that is crucial for controlling feeding, metabolism and body weight in ruminants. Leptin is mainly produced and secreted by adipocytes. The secretion of leptin may be in relation to fat stores. Body fatness strongly regulates leptin and its responses to other factors. For example, leptinemia is higher after underfeeding or during lactation in fat than in lean animals. Leptin is reduced during periods of under nutrition. Skeletal muscles and bone may also release the hormone. Leptin is mainly involved in metabolic homeostasis or delivering information to the hypothalamus regarding the amount of energy stores, the hypothalamus then alters the central nervous system function and regulates the synthesis of glucocorticoids and insulin as well as food intake and energy storage. Leptin plays a primary role in the regulation of glucose homeostasis through the central nervous system. Leptin also acts as a signal of nutritional status.

The skeletal system in ruminants is a major site of insulin disposal. Leptin may enhance the glucose uptake in the skeletal muscle and insulin signaling. Leptin may also improve insulin sensitivity in the liver. Leptin exerts dual effects depending on bone tissue, skeletal maturity and/or the signaling pathway. During skeletal development, leptin stimulates bone growth. Growth hormone (GH) is secreted by the pituitary and acts on peripheral tissues through the simulation of IGF-1 synthesis and secretion. The GH and IGF-1 axis have pleiotropic effects on the skeleton by influencing bone formation and resorption. GH stimulates osteoblastogenesis and bone formation. Leptin influences GH regulation and secretion by activating neuronal nitric oxide synthase enzyme. GH then induces the liver to produce IGF-1. IGF-1 stimulates osteoblastic activity thereby promoting skeletal growth.

When ruminants are growing, variations in nutrition impact developmental processes that could be regulated by leptin. For example, high levels of nutrition lead to early onset of puberty and a younger age of calving. However, this early onset of puberty is associated with decreased parenchymal mammary development and decreased future milk yield. By feeding the ruminant a feed composition including at least hydroponically sprouted cereal grains, nutritional levels are managed while improving skeletal development while at least maintaining future milk yield.

### Exemplary Methods

To investigate the hydroponically sprouted cereal grains as a sustainable and scalable solution to improve skeletal development in prepubescent dairy heifers, a replicated pen study was performed investigating the influence of hydroponically sprouted cereal grains (HG) on 80 post weaned jersey heifers averaging 80 days of age and 80.7 kg in body weight and 89 cm in wither height. Heifers were assigned to one of eight identical pens and delivered either a control or hydroponically sprouted cereal grains treatment diet in a commercial setting. Dietary treatments consisted of a control and treatment group (11% HG; DM basis), randomly assigned and delivered continuously for a six-week comparison period. Dry matter intake, apparent nutrient digestibility, blood serum chemistry, wither height, body length, and weight gain were evaluated during the observation. Growth was measured through weight, wither height, and length along the medial plane from the shoulders to the coccygeal vertebrae. Pens were administered dietary treatments ad liberum with dry matter intake and refusal weight recorded weekly. Apparent nutrient digestibility was assessed through weekly paired total mixed rations (TMR) and manure sampling for analysis using the Combs-Goeser in vitro digestion technique (Goeser and Combs, 2009). Blood serum was collected from the lateral vein at approximately 140 days of age for analysis. Daily rate of gain, feed conversion ratio, and cost of gain was assessed through monthly weight evaluations. Blood serum chemistry was evaluated through individual animal blood serum samples collected from the lateral tail vein.

### Exemplary Results

Values reported in the experiment support the importance of stimulating metabolizable energy intake of the post weaned animal. Statistically significant (p<0.05) effects were identified for neutral detergent fiber (NDF) digestion, wither height and cost of gain with trending (p<0.2) significance observed for dry matter intake, body weight, body length, average daily rate of gain, and feed efficiency, as shown in Tables 1-3.

TABLE 1
Statistical analysis of nutrient digestion, dry matter intake (DMI), growth, average
daily gain (ADG), feed efficiency, and cost of gain.

| | HG | Control | SEM | DF | Contrast | p-value | Unit |
|---|---|---|---|---|---|---|---|
| Organic Matter | 68.9 | 69.9 | 2.3 | 7 | −1.0 | 0.786 | % OM |
| (OM) | | | | | | | |
| Digestibility | | | | | | | |
| NDF | 45.7 | 44.9 | 2.4 | 7 | 0.8 | 0.006 | % NDF |
| Digestibility | | | | | | | |
| DMI | 3.13 | 2.95 | 0.09 | 7 | 0.18 | 0.115 | kg day⁻¹ |
| Weight | 124.2 | 122.5 | 1.2 | 7 | 6.2 | 0.102 | kg |
| Height | 100.3 | 96.5 | 0.2 | 7 | 3.8 | 0.003 | cm |
| Length | 87.2 | 84.9 | 0.44 | 7 | 2.3 | 0.165 | cm |
| ADG | 1.22 | 1.10 | 0.03 | 7 | 0.06 | 0.102 | kg day⁻¹ |
| Feed | 0.20 | 0.17 | 0.01 | 7 | 0.03 | 0.172 | kg MJ⁻¹ |
| Efficiency | | | | | | | |
| Cost of | 0.21 | 0.32 | 0.01 | 7 | −0.11 | 0.009 | $ kg⁻¹ |
| Gain | | | | | | | |

SEM = Standard error of the mean, DF = degrees of freedom

TABLE 2
Statistical analysis of nutrient digestion, DMI, growth, ADG, feed efficiency, and cost of gain.

| | HG | Control | SEM | DF | Contrast | p-value | Unit |
|---|---|---|---|---|---|---|---|
| OM | 68.4 | 66.8 | 2.5 | 7 | 1.6 | 0.512 | % OM |
| Digestibility | | | | | | | |
| NDF | 53.1 | 40.7 | 3.7 | 7 | 12.4 | 0.026 | % NDF |
| Digestibility | | | | | | | |
| DMI | 3.12 | 2.93 | 0.08 | 7 | 0.19 | 0.081 | kg day⁻¹ |
| Weight | 124.9 | 122.9 | 2.5 | 7 | 2.0 | 0.613 | kg |
| Height | 100.3 | 96.6 | 0.6 | 7 | 3.7 | 0.006 | cm |
| Length | 87.2 | 84.9 | 0.4 | 7 | 2.3 | 0.165 | cm |
| ADG | 1.3 | 1.08 | 0.07 | 7 | 0.05 | 0.613 | kg day⁻¹ |
| Feed | 0.35 | 0.34 | 0.02 | 7 | 0.01 | 0.883 | ADG |
| Efficiency | | | | | | | DMI⁻¹ |
| MJ | 12.2 | 14.2 | 1.0 | 7 | −2.1 | 0.177 | MJ kg⁻¹ |
| Efficiency | | | | | | | |
| Cost of Gain | 0.24 | 0.34 | 0.02 | 7 | −0.10 | 0.017 | $ kg⁻¹ |

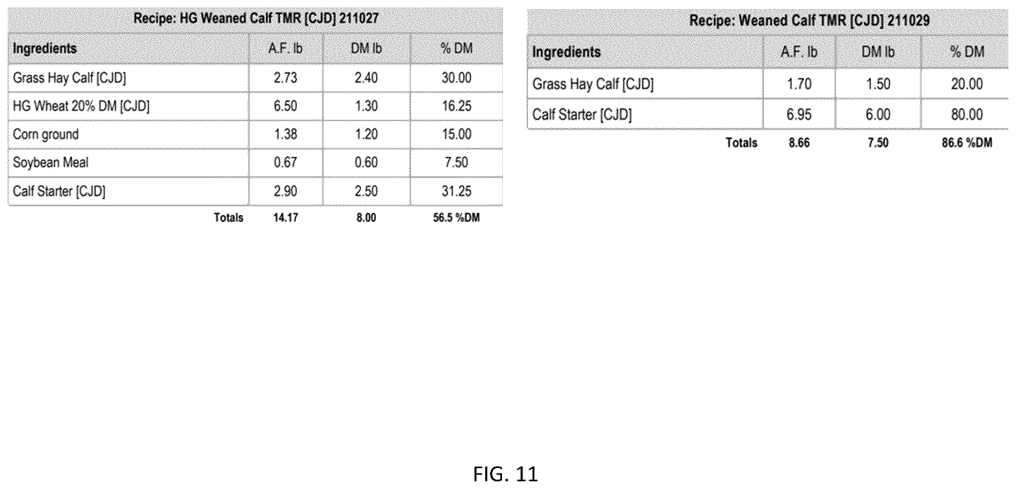

With significantly lower energy density supplied in the HG treatment, positive changes to skeletal development and performance are further emphasized in FIG. 11 and Tables 3 and 4. In alignment with performance increases, significant alterations to blood serum anion gap and blood serum glucose were reported with positive trends identified for blood urea nitrogen, total carbon dioxide, and total bilirubin levels.

TABLE 3
Statistical analysis of blood serum chemistry analysis including blood
urea nitrogen (BUN) and total dissolved carbon dioxide (TCO₂).

| | HG | Control | SEM | DF | Contrast | p-value | Unit |
|---|---|---|---|---|---|---|---|
| BUN | 16.1 | 13.7 | 0.81 | 7 | 2.4 | 0.095 | mg dL⁻¹ |
| TCO² | 33.5 | 31.0 | 0.72 | 7 | 2.5 | 0.071 | mmol L⁻¹ |
| Anion Gap | 17.5 | 19.7 | 0.56 | 7 | −2.2 | 0.041 | mmol L⁻¹ |
| Glucose | 94.0 | 80.8 | 1.95 | 7 | 13.0 | 0.006 | mg dL⁻¹ |
| Total | 0.13 | 0.19 | 0.02 | 7 | −0.06 | 0.075 | mg dL⁻¹ |
| Bilirubin | | | | | | | |

TABLE 4
Statistical analysis of dietary treatment nutrition analysis including dry matter (DM), crude
protein (CP), acid detergent fiber (ADF), neutral detergent fiber (NDF), and water-soluble
carbohydrates (WSC), net energy of gain (NEG), and net energy of maintenance (NEM).

| | HG | Control | SEM | DF | Contrast | p-value | Unit |
|---|---|---|---|---|---|---|---|
| DM | 62.6 | 79.3 | 3.0 | 26 | −16.7 | <0.00 | % |
| CP | 18.7 | 21.5 | 0.5 | 26 | −2.8 | <0.00 | % DM |
| ADF | 17.8 | 11.0 | 1.2 | 10 | 6.8 | 0.007 | % DM |
| NDF | 32.0 | 23.4 | 1.3 | 26 | 8.5 | <0.00 | % DM |
| Lignin | 4.3 | 4.0 | 0.1 | 10 | 0.3 | 0.107 | % DM |
| Starch | 20.8 | 25.1 | 1.1 | 26 | −4.4 | 0.011 | % DM |
| WSC | 10.9 | 10.7 | 0.1 | 10 | 0.2 | 0.331 | % DM |
| NEG | 4.3 | 5.0 | 0.1 | 10 | −0.7 | 0.009 | MJ kg⁻¹ |
| NEM | 6.9 | 7.5 | 0.1 | 10 | −0.7 | 0.009 | MJ kg⁻¹ |

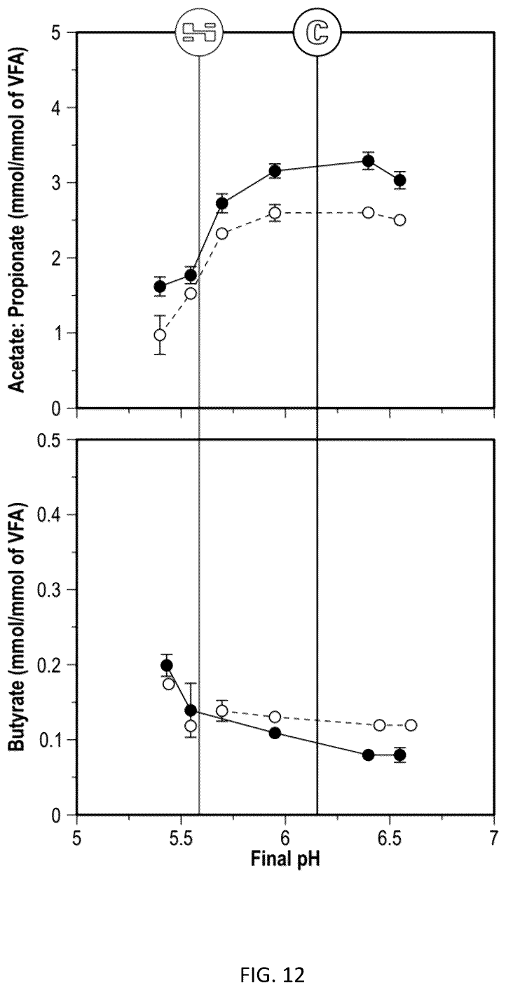

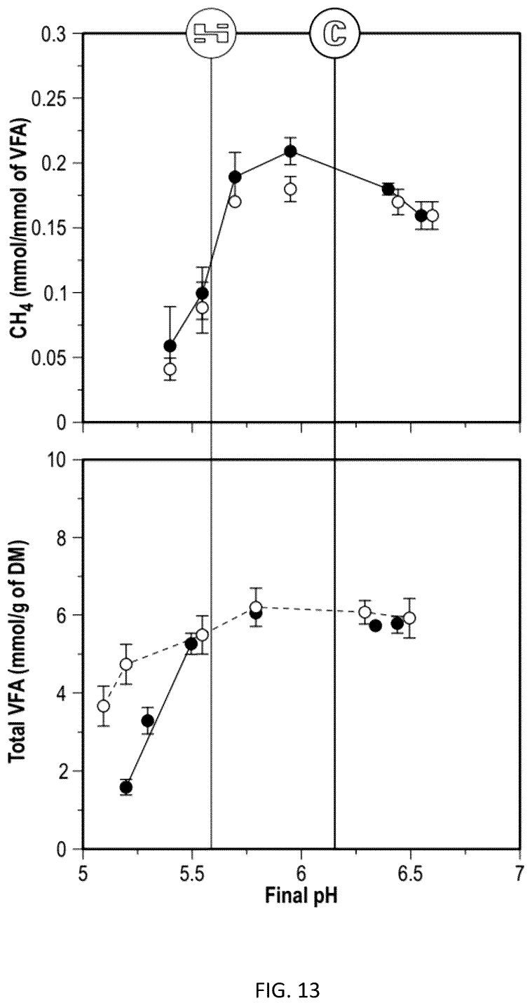

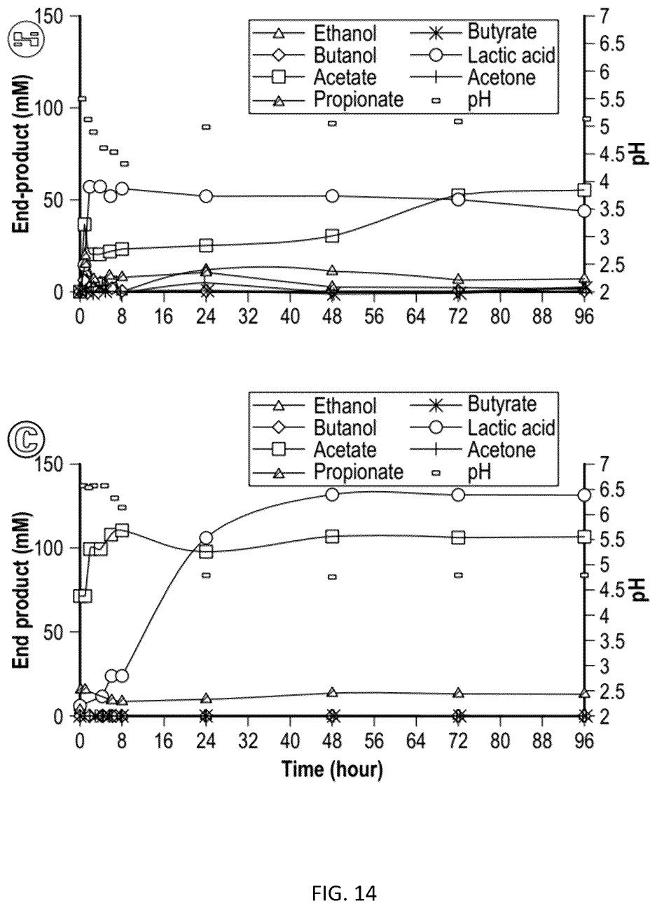

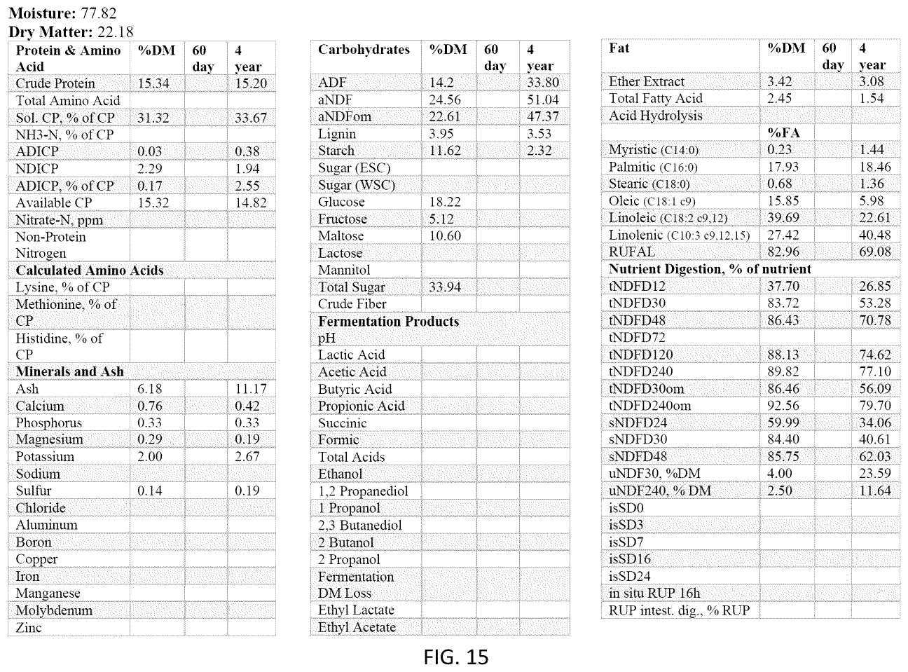

Hydroponically sprouted cereal grains introduced at a 14% dry matter inclusion level thereby altering glucose, propionate, acetate, butyrate, and hexose concentrations as shown in FIGS. 12-14. FIG. 15 illustrates the nutritional breakdown of the hydroponically sprouted grains in accordance with an exemplary aspect of the present disclosure. Hydroponically sprouted grains may also be introduced at a higher percentage or a lower percentage, such as 10%, 12%, 16% 20%, 25%, or 50% or even higher. The percentage of hydroponically sprouted grains may be selected based on the ruminants. In other aspects of the present disclosure, the percentage of hydroponically sprouted grains may increase over a period of time.

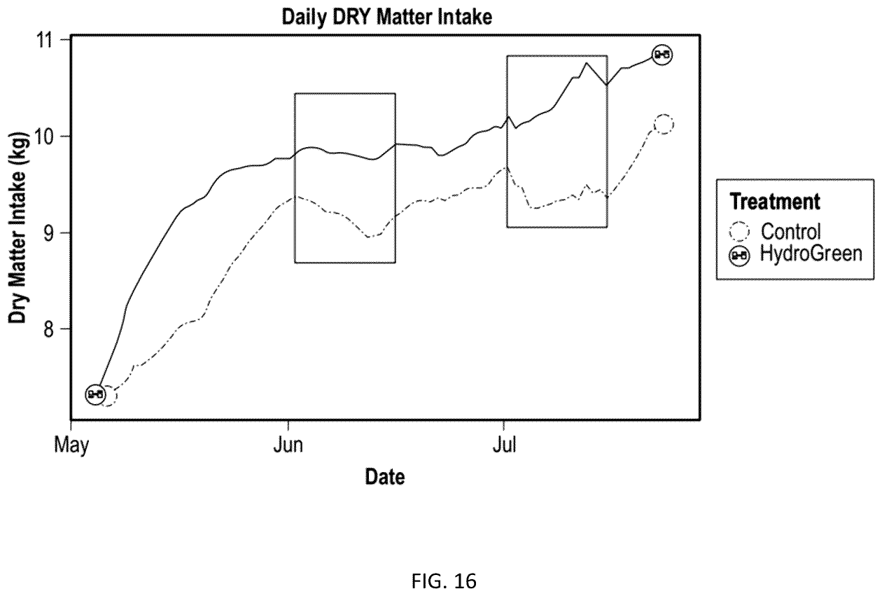

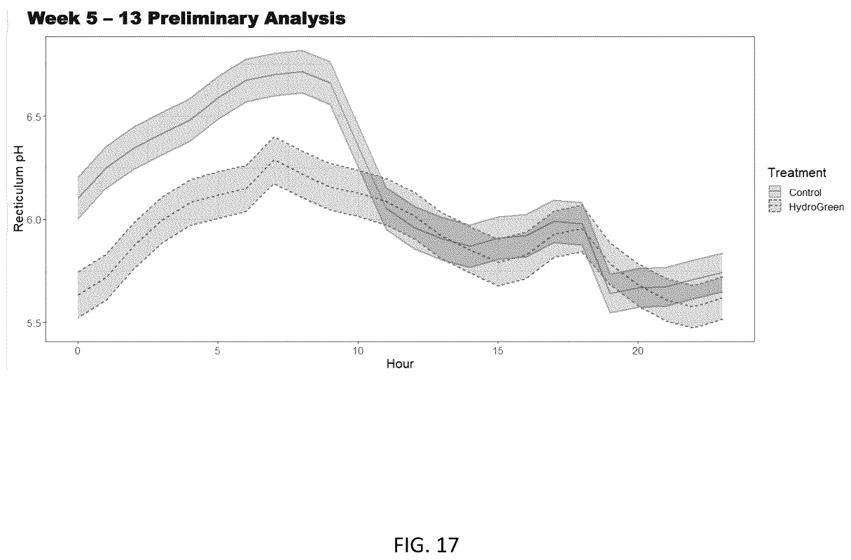

Inclusion of hydroponically sprouted grains resulted in significant improvements in wither height and NDF digestibility, along with positive trends in body length and intake. Increased wither height estimated increase in first lactation milk production by 5% ($194 cow⁻¹ year⁻¹). Positive changes in skeletal development coupled with significant reductions in feed cost illustrate the value of incorporating hydroponically sprouted cereal grains into transitional calf diets. These findings highlight the feeding value of hydroponically sprouted cereal grains as a novel local chain agricultural technology solution. In some aspects of the present disclosure, there was an 8 percent increase in average daily weight gain, 30% increase in liver health and function (blood serum bilirubin) and/or a 25% reduction in the cost of weight gain. In some aspects of the present disclosure there may be at least a 5 to 15% feed efficiency increase or at least a 10% increase in fiber digestion. In some aspects of the present disclosure, there is an increase in dry matter intake, as shown in FIG. 16. In some aspects of the present disclosure there may be a 95% increase in water savings. In other aspects of the present disclosure, the percentages may increase or decrease. More stabilized pH levels were observed translating to a homeostasis of energy balance, as shown in FIG. 17.

Estimated wither height changes have been previously reported to be correlated to an average 5% increase in first lactation milk production (Bar-Peled et al., 1997). Supporting the importance of IGF-1 on early growth and development, blood serum glucose levels were identified to increase 16% in the HG treatment. Aligned with the theory of leptin's involvement in IGF-1 and early growth and development total bilirubin were reduced 32% highlighting the potential link of blood serum glucose, IGF-1, leptin, and liver function (Brown et al., 2005). Elevated BUN levels with lower crude protein supplied in the dietary treatment highlight a valuable increase in protein utilization and microbial protein synthesis in the developmental stage (Brown et al., 2005).

Through increasing relative concentration of hexoses and in turn propionate, gluconeogenesis, and blood serum glucose and insulin levels, hydroponically sprouted cereal grains may be used as an approach to positively impact not only energy balance but improve nutrition and skeletal development. Hydrolysis reactions external to the rumen, e.g., enzymatic hydrolysis of hemicelluloses during germination; improve efficiency of energy utilization, reduce hydrogen dioxide and carbon dioxide formation within the rumen improving feed efficiency, lower the acetate to propionate volatile fatty acid ratio supporting positive progesterone profile shifts, improve gluconeogenesis supporting improved energy balance, and support the development of reproductive structures within the animal. Adding a nutritious hydroponically sprouted cereal grains to the feed composition allows complex carbohydrates to be more easily converted into digestible sugars and fibers, enabling better ration digestibility in the rumen and improved rumen health. Hydrolytic enzymes naturally occurring in the fresh plant enhance the digestibility and absorption of nutrients like vitamins and minerals from the ration as well as the overall diet, which can help prevent serious and costly metabolic disorders.

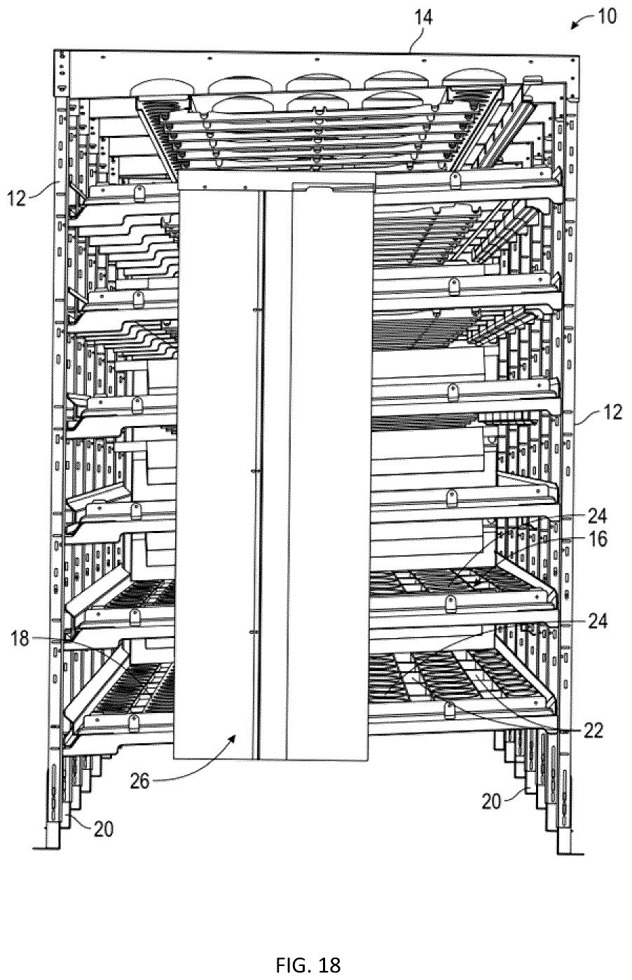

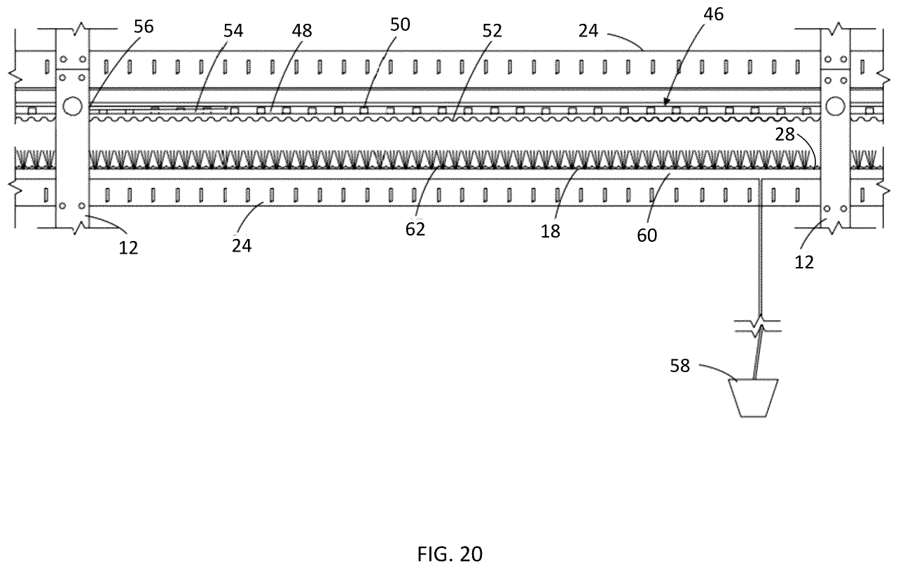

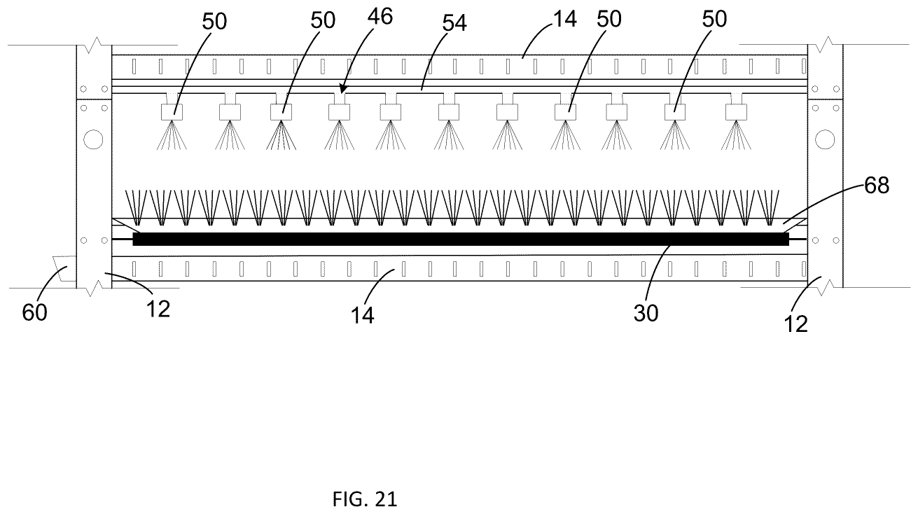

FIG. 18 illustrates a grower system 10. The grower system 10 can provide aerobic conditions allowing the plant to increase dry matter, maximize enzyme activity and increase sugar concentration. The grower system 10, shown in FIGS. 18-22 may include a plurality of vertical members 12 and a plurality of horizontal members 14 removably interconnected to form an upstanding seed growing table 16 with one or more seed beds 18. In some aspects of the present disclosure, the grower system 10 may have one or more seed beds 18. Each vertical member 12 can be configured to terminate at the bottom in an adjustable height foot 20. Each foot 20 can be adjusted to change the relative vertical position or height of one vertical member 12 relative to another vertical number 12 of the seed growing table 16. The horizontal member 14 can be configured to include one or more lateral members removably interconnected with one or more longitudinal members 24. A pair of vertical members 12 may be separated laterally by a lateral member 22 thereby defining the width or depth of the seed growing table 16. Longitudinal members 24 may be removably interconnected with lateral members 22 by one or more connectors 26.

Each seed bed 18 may include a seed belt 28, such as a seed film, operably supported by seed growing table 16. Seed belt 28 can be configured according to the width/depth of seed growing table 16. By way of example, the width/depth of seed belt 28 can be altered according to changes in the width/depth of seed growing table 16. The seed belt 28 material can be hydrophobic, semi-hydrophobic or permeable to liquid. In at least one aspect, a hydrophobic material may be employed to keep liquid atop the seed belt 28. In another aspect, a permeable or semi-permeable material can be employed to allow liquid to pass through the seed belt 28. Advantages and disadvantages of both are discussed herein. Traditional pans use hydrophobic material as part of the seed bed. This may increase water stress as water stays within the seed bed for prolonged periods, creating hypoxic conditions and increasing the concentration of abscisic acid (ABA). The seeds use up the available oxygen. In one aspect, seed belt 28 may be discontinuous and may have separate or separated terminal ends. The seed belt 28 may have a length of at least the length of the seed bed 18 and generally a width of the seed bed 18 and may be configured to provide a seed bed for carrying seed. The seed belt 28 may be configured to move across the seed bed 18. Seed belt 28 may also rest upon and slide on top of horizontal members 14. One or more skids or skid plates (not shown) may be disposed between seed belt 28 and horizontal members 14 to allow seed belt 28 to slide atop horizontal members 14 without binding up or getting stuck. The seed bed 18 or seed belt 28 may be positioned at a slope to encourage the drainage of water facilitating an increased oxygenated environment when compared to a pan type fodder set up.

To provide room for expansion the seed belt 28 or seed bed 18 may have a seed egress 68 on one or more sides of the seed bed 18, such as a first side 70 and an opposing second side 72. The seed egress 68 allows room for expansion as the seeds grow, lessening the growth compression of the seeds. If the seed bed 18 has walls on the first side 70 or the second side 72, the walls may prevent the seeds from expanding thereby compressing some or all of the seeds. The compressed seeds may receive little to no oxygen resulting in hypoxic or anaerobic conditions. The seed egress 68 may not be covered with seeds during seed out. The empty space allows for expansion as the seed doubles in volume in the first few growth stages, such as in the first 24 hours. If the seeds do not have room to expand, the seed may be subjected to a dense environment with reduced heat, water, and oxygen exchange capabilities.

Each seed bed 18 may include a liquid applicator 46 operably configured atop each seed bed 18 for irrigating seed disposed atop each seed bed 18. The seed may be irrigated with water. The dimensions of the seed bed 18 may be configured to accommodate need, desired plant output, or maximization of enzyme activity. Liquid applicator 46 may be configured adjacent at least one longitudinal edge of seed bed 18. Liquid applicator 46 may also be operably configured adjacent at least one lateral edge of seed bed 18. Preferably, liquid applicator 46 may be configured adjacent a longitudinal edge of seed bed 18 to thereby provide drip-flood irrigation to seed bed 18 and seed 74 disposed atop seed bed 18. Liquid applicator 46 may include a liquid guide 48 and liquid distributor 50 with a liquid egress 52 having a generally undulated profile, such as a sawtooth or wavy profile generally providing peak (higher elevated) and valley (lower elevated) portions. Liquid applicator 46 can include a liquid line 54 configured to carry liquid 62 from a liquid source 56, such as a liquid collector 58 or plumbed liquid source 56. Liquid 62 may exit liquid line 54 through one or more openings and may be captured upon exiting liquid line 54 by liquid guide 48 and liquid distributor 50. The one or more openings in liquid line 54 can be configured as liquid drippers, intermittently dripping a known or quantifiable amount of liquid 62 over a set timeframe into liquid guide 48. The one or more openings may be configured intermittently along a length of liquid line 54 or dispersed in groupings along a length of liquid line 54. The one or more openings in liquid line 54 can be operably configured to equally distribute the liquid 62 down the seed bed 18 and slowly drip liquid into the seed bed 18. Drip or flood irrigating the growing surface provides a layer of liquid 62 for soaking the seed and can provide liquid 62 to seed 74 on seed bed 18 in a controlled, even distributive flow. Liquid distributor 50 can be configured with a liquid guide 48 adapted to collect liquid 62 as it exits liquid line 54. Collected liquid may be evenly distributed by liquid distributor 50 and exit the liquid distributor 50 onto the seed bed 18 via the liquid egress 52.

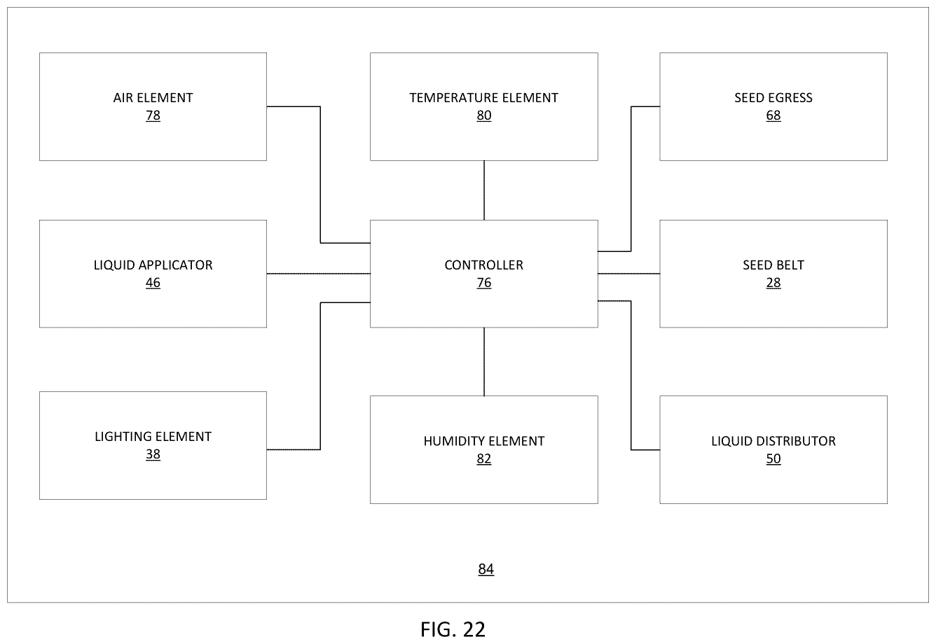

According to at least one aspect, liquid 62 egressing from liquid distributor 50 may travel atop seed belt 28 beneath. In another aspects of the present disclosure liquid may travel between the hydroponically sprouted cereal grains or seeds 74 atop seed bed 18 as shown in FIG. 22. Other configurations of liquid applicator 46 are also contemplated herein. For example, in one aspect, liquid 62 may enter liquid applicator 46 through a liquid line 54 and exit liquid line 54 through a plurality of openings. Liquid 62 from liquid line 54 may coalesce into a small reservoir creating a balanced distribution of liquid 62 across a length of liquid distributor 50. When this small reservoir becomes full, the liquid 62 may run over and out of liquid egress 52, such as between the teeth of liquid egress 52. In this manner, liquid 62 may be equally distributed down an entire length and across an entire width of the seed bed 18. From liquid egress 52, liquid 62 may drip onto a seed belt 28 where it may run under a bulk of hydroponically sprouted cereal grains 74 on the seed belt 28 to soak or make contact with the seed. The root system of hydroponically sprouted cereal grains 74 on the seed belt 28, along with a wicking effect, may move the liquid 62 up through the hydroponically sprouted cereal grains 74 to water all the seeds and/or plants.

Liquid applicator 46 may be disposed atop each seed bed 18. Liquid applicator 46 may include a plurality of liquid distributors 50 operably configured in a liquid line 54 operably plumbed to a liquid source 56. Liquid distributor 50 can include spray heads, such as single or dual-band spray heads/tips, for spray irrigating hydroponically sprouted cereal grains 74 disposed atop each seed bed 18. In one aspect, a plurality of liquid lines 54 may be disposed in a spaced arrangement atop each seed bed 18. Each liquid line 54 may traverse the length of the holding container and may be plumbed into connection with liquid source 56. Other liquid lines 54 can be configured to traverse the width of seed bed 18. Liquid 62 may be discharged from each liquid distributor 50 for spray irrigating hydroponically sprouted cereal grains 74 atop each seed bed 18. In another aspect, each liquid line 54 may be oscillated back and forth over a 10°, 15°, 20°, 25°, 35°, 40°, 45°, or greater radius of travel for covering the entire surface area of the hydroponically sprouted cereal grains 74 atop each seed bed 18. In the case where dual angle spray heads may be used for liquid distributor 50, the oscillation travel of each liquid line 54 can be reduced thereby reducing friction and wear and tear on liquid applicator 46. The process of applying liquid to the hydroponically sprouted cereal grains 74 or plant can be automated by a controller 76, graphical user interface, and/or remote control. A drive mechanism can be operably connected to each liquid line 54 for oscillating or rotating each line through a radius of travel. Liquid applicator 46 can be operated manually or automatically using one or more controllers operated by a control system 84.

Liquid applicator 46 may be configured to clean seed bed 18 of debris, contaminants, mold, fungi, bacteria, and other foreign/unwanted materials. Liquid applicator 46 can also be used to irrigate hydroponically sprouted cereal grains 74 with a disinfectant, nutrients, or reactive oxygen species as hydroponically sprouted cereal grains 74 is released onto seed bed 18 from a seed dispenser. A time delay can be used to allow the reactive oxygen species or nutrients to remain on hydroponically sprouted cereal grains 74 for a desired time before applying or irrigating with fresh water. The process of cleaning, descaling, and disinfecting seed bed 18 using liquid applicator 46 can be automated by a controller 76, graphical user interface, remote control and/or the control system 84.

Liquid applicator 46 can be operated immediately after seeding of the seed bed 18 to saturate hydroponically sprouted cereal grains 74 with liquid. Hydroponically sprouted cereal grains 74 in early, mid, and late stages of growth can be irrigated with liquid 62 using liquid applicator 46. Liquid applicators 46 can be operated simultaneously, intermittently, alternately, and independent of each other. During early stages of hydroponically sprouted cereal grains 74 growth, both liquid applicators can be operated to best saturate hydroponically sprouted cereal grains 74 to promote sprouting and germination. During later stages of growth, a first liquid applicator 46 can be used to irrigate more than a second liquid applicator 46, depending upon saturation level of hydroponically sprouted cereal grains 74 growth. Liquid applicator 46 can be operated during seeding of seed bed 18 and movement of seed bed 18 in the second direction to spray hydroponically sprouted cereal grains 74 dispensed atop seed bed 18 to saturate hydroponically sprouted cereal grains 74 with liquid. The liquid provided to liquid applicators 46 could include additives such as disinfectants, reactive oxygen species, fertilizer, and/or nutrients. Nutrients, such as commonly known plant nutrients such as calcium and magnesium, can be added to liquid dispensed from liquid applicators 46 to promote growth of healthy plants and/or increase the presence of desired nutrients in harvested seed. Liquid applicators 46 can be used also to sanitize seed bed 18 before and/or after winding on or unwinding of the seed belt, the seed bed 18, or seed egress 68 of the seed belt.

Liquid distributors 50 and their various components, along with other components of the grower system 10, can be sanitized by including one or more disinfectants, such as reactive oxygen species used by each liquid distributor 50. For example, liquid guide 48, liquid lines 54, liquid egress 52, drain trough 60, liquid collector 58, seed bed 18, liquid distributors 50, and other components of the growing system may be sanitized. In another aspect, liquid applicators 46 can be used to clean and sanitize seed bed 18 before, between, or after seeding and harvesting. A separate liquid distributor or manifold can be configured to disinfect or sanitize any components of the growing system that carry liquid for irrigation and cutting or receive irrigation or cutting runoff from the one or more holding containers.

The liquid 62 may be constantly applied, or the applicator may apply the liquid 62 at a set time frame or at a quantifiable amount. For example, the liquid applicator 46 may apply the liquid 62 for a first time period such as 1 minute and then the liquid applicator may stop applying the liquid 62 for a second time period, such as 4 minutes, or 1 min of liquid application for every 5 minutes. The cycle may continue until the developmental phase or seed out phase terminates. In another example, the liquid 62 may be applied for 10 min every 2 hours. The liquid applicator 46 may provide a controlled, evenly distributed flow allowing the liquid 62 to reach a maximum number of seeds. Excess liquid 62 may be captured, recycled, and reused by the grower system 10. If the seed bed 18 has an egress or a slant, the slant may aid in the even distribution of the liquid as it egresses through the seed bed 18. In some aspects, the liquid applicator 46 may guide the distribution of the liquid to areas within the seed bed 18, a portion of the seeds, or a portion of the plants that need more application. The liquid applicators 46 may also oscillate to cover the larger areas of the seed bed 18 or the entire length and width of the seed bed 18 or seed belt 28.

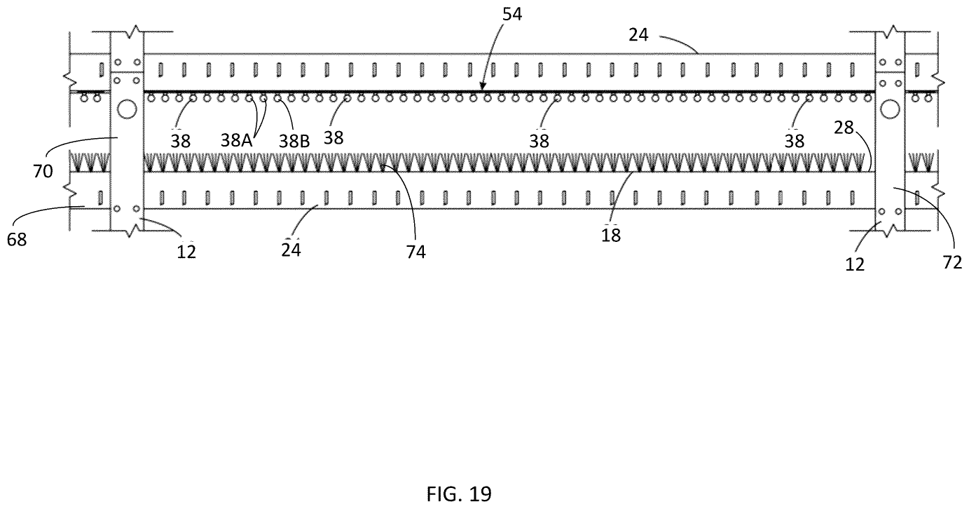

Each seed bed 18 may include one or more lighting elements 38 or housing lights for illuminating hydroponically sprouted cereal grains 74 atop seed belt 28 to facilitate hydroponic growth of hydroponically sprouted cereal grains 74 atop seed belt 28, as shown in FIG. 19. Lighting elements 38 may be operably positioned directly/indirectly above each seed bed 18. Lighting elements 38 can be turned off and on for each level using a controller 76 or the control system 84. In some aspects, one level of the grower system 10 may have the lighting elements 38 turned on while another level may have the lighting elements turned off. Lighting elements 38 can be powered by an electrochemical source or power storage device, electrical outlet, and/or solar power. In one aspect, lighting elements 38 may be powered with direct current power. Contemplated lighting elements 38 include, for example, halide, sodium, fluorescent, and LED strips/panels/ropes, but are not limited to those expressly provided herein. One or more reflectors (not shown) can be employed to redirect light from a remote source not disposed above each seed bed 18. Lighting elements 38 can be operably controlled by a controller, a timer, user interface, remotely or through the control system 84. Operation of lighting elements 38 can be triggered by one or more operations of grower system 10. For example, operation of a seed belt 28 can trigger operation of lighting elements 38. The process of lighting a seed bed 18 can be automated by controller, graphical user interface, and/or remote control. In one aspect, lighting elements 38 may be low heat emission, full ultraviolet (UV) spectrum, light emitting diodes that are cycled off and on with a controller, preferably on 18 hours and off 6 hours in a 24-hour period. The lighting elements 38 may emit multiple light frequencies at a specific ratio, such as a 2:1 ratio of R/FR light or the lighting elements may emit different light frequencies. For example, lighting element 38A may emit red light and lighting element 38B may emit white light. The lighting elements may be placed sequentially or in a pattern that permits the proper ratio. The controller or control system 84 may control the frequency, intensity, or ratio of the lighting elements 38.

The grower system 10 may have a control system 84 for controlling different environmental conditions or operating conditions of the grower system. The control system 84 may control at least one air element 78 such as a fan or HVAC system to control air movement around the seed bed, as shown in FIG. 22. The air element 78 may be operably connected to the controller 76. A room or environment where the grower system 10 may be stored may also have one or more fans used to control air movement. The air movement or flow may be changed depending on the developmental phase of the hydroponically sprouted cereal grains 74 on the seed bed. A temperature element 80, such as an HVAC unit, may be operably connected to the grower system 10, controller 76, or the seed bed 18 to control the temperature of the environment of the seed bed 18. The temperature element 80 may maintain temperatures ranging of 65 to 85 degrees Fahrenheit or 18 to 30 degrees Celsius. A humidity element 82 may be operably connected to the controller, the control system 84, growing system 10, or seed bed 18 for controlling the humidity of the environment of the seed bed 18. The humidity element 82 may maintain a relative humidity level between 50% and 90%. The temperature element 80, air element 78, and humidity element 82 may all include the same HVAC unit. The temperature and air humidity may be changed depending on the developmental phase of the hydroponically sprouted cereal grains 74 on the seed bed 18. The process of controlling the air movement, temperature, and humidity of a seed bed 18 can be automated by controller, graphical user interface, remote control, and/or the control system. The lighting, temperature, air flow, and liquid application may all affect the humidity of the seed bed 18.

In hydroponic growing systems 10, plants may be suspended in water full-time or fed by an intermittent flow of water. Aeroponic plants are never placed into water, instead being given nutrients from a mist that's sprayed onto their roots. In some aspects of the present disclosure, the grower system 10 may be an aeroponic system. Aeroponic plants are suspended in mid-air. The grower system 10 may have a perforated platform or tower. Seedlings may be planted in the holes or perforations of the platform. The root systems of the seedlings may be enclosed in a root chamber. Inside the root chamber may be a series of spray nozzles that pull nutrient-rich water from a reservoir. The nozzles may routinely spray the roots with an ultra-fine mist of the water solution. The grower system 10 may be a closed loop system. Low-pressure aeroponic setups use a coarser mist than high-pressure systems to hydrate plants. High-pressure aeroponic systems atomize the nutrient liquid into an ultra-fine mist, supplying the root systems with nutrients, water, and oxygen more efficiently than low-pressure systems. Since aeroponics is soilless, the roots receive direct nutrient delivery. The grower system 10 may include a growth tray for housing the plants. The grower system may have a water reservoir and a nutrient reservoir for holding water and nutrient solutions. The water reservoir and nutrient reservoir may be combined. The water and nutrients may be transferred from the reservoir to the plants via liquid applicators, such as nozzles. A pump may be used to transfer the nutrients and water from the reservoirs to the nozzles. The pump may be submersible. Grow lights may be used to aid in the growth of the plants. In some aspects of the present disclosure, the roots may be housed in the container and not exposed to the grow lights, while the stem and leaves of the plant are housed outside the container with the stem growing through the perforations and leaves are exposed to the grow lights. An air pump may be used to provide additional aeration and ensure there is enough carbon dioxide within the grower system 10. If the grower system 10 uses aeroponics to grow the hydroponically sprouted cereal grains the grower system may utilize an enclosed and completely controlled environment. The plants are contained in a humid atmosphere where frequent misting delivers a nutrient rich solution right to the roots, keeping the crop from drying out. Because the entire process is enclosed, the mist is able to remain around the plants for longer, helping them grow much quicker than in a traditional outdoor farm.

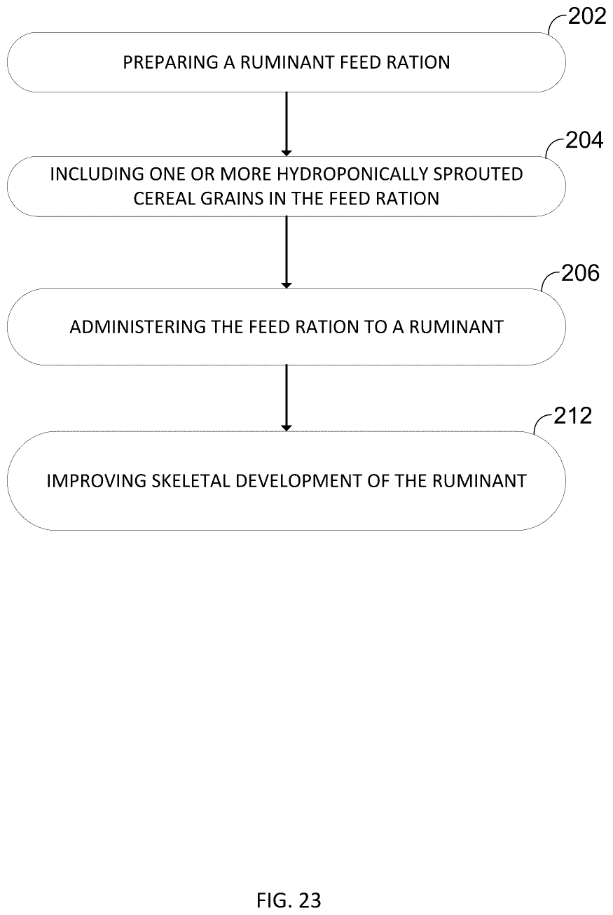

In at least one aspect of the present disclosure a method for improving skeletal developments is shown in FIG. 23. The method may include, for example, such steps as preparing a ruminant feed ration (Step 202), including one or more hydroponically sprouted cereal grains in the feed ration (Step 204), administering to a ruminant the feed ration (Step 206) having at least one feed component that includes one or more hydroponically sprouted cereal grains for improving skeletal development in the ruminant (Step 212). The feed ration may increase dry matter intake of the ruminant. The fertility rates may be increased by increasing leptin levels, growth hormone levels, or insulin growth factor 1 levels in the ruminant with the feed ration. The increase in leptin levels, growth hormone, or insulin growth factor 1 may stimulate skeletal development. The feed ration may increase crude protein uptake by the ruminant. The method may further include increasing nutrition levels within the ruminant with the feed rations, wherein the increased nutritional levels improve skeletal development. The method may further include reducing dihydrogen concentrations within the rumen with the feed ration. The method may also include lowering the acetate to propionate volatile fatty acid ratio and increasing glucose within the feed ration. In other exemplary aspects of the present disclosure the steps may include increasing metabolic energy in the ruminant with the feed ration, increasing gluconeogenesis in the ruminant with the feed ration, wherein the increased gluconeogenesis improves energy balance in the ruminant. The method may also include improving the height, weight or length of a ruminant or a prepubescent ruminant.

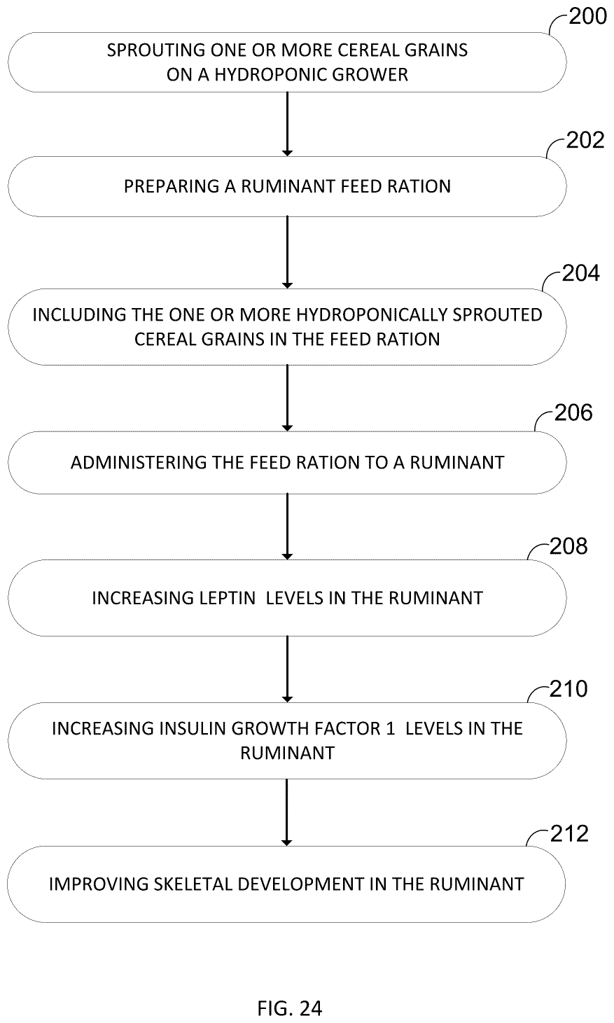

In another aspect of the present disclosure a method for improving skeletal development is disclosed and shown in FIG. 24. The method may include steps, such as, for example, sprouting one or more cereal grains on a hydroponic grower (Step 200) for preparing a ruminant feed ration (Step 202) by including the one or more hydroponically sprouted cereal grains in the feed ration (Step 204), administering the feed ration to a ruminant (Step 206), increasing leptin levels within the ruminant (Step 208), increasing insulin growth factor 1 levels in the ruminant (Step 210) and improving skeletal development of the ruminant (Step 212). The increase in insulin may levels supports the improvement of skeletal development within the ruminant.

The disclosure is not to be limited to the particular aspects described herein. In particular, the disclosure contemplates numerous variations in improving skeletal development in ruminants. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of aspects, processes or methods of the disclosure. It is understood that any other modifications, substitutions, and/or additions can be made, which are within the intended spirit and scope of the disclosure.

What is claimed is:

1. A method for hydroponically sprouted cereal grains as a mechanism for improving skeletal development in ruminants, the method comprising:
administering to a ruminant a feed ration having at least one feed component comprising one or more hydroponically sprouted cereal grains for improving skeletal developments in a ruminant.

2. The method of claim 1, further comprising:
increasing dry matter intake of the ruminant with the feed ration.

3. The method of claim 1, further comprising:
increasing crude protein intake of the ruminant with the feed ration.

4. The method of claim 1, further comprising:
reducing dihydrogen concentrations within the rumen with the feed ration;
lowering the acetate to propionate volatile fatty acid ratio; and
increasing glucose within the feed ration.

5. The method of claim 1, further comprising:
increasing metabolic energy in the ruminant with the feed ration.

6. The method of claim 1, further comprising:
increasing insulin growth factor 1 levels.

7. The method of claim 1, further comprising:
increasing leptin levels within the ruminant with the feed ration.

8. The method of claim 1, further comprising:
increasing gluconeogenesis in the ruminant with the feed ration, wherein the increased gluconeogenesis improves energy balance in the ruminant.

9. The method of claim 1, further comprising:
increasing nutrition levels within the ruminant with the feed rations, wherein the increased nutritional levels improve skeletal development.

10. A method for improving skeletal development in a ruminant, the method comprising:
hydroponically sprouting one or more cereal grains for at least one feed component for ruminants;
feeding the ruminant a feed ration having the at least one feed component comprising the one or more hydroponically sprouted cereal grains for improving skeletal development in the ruminant;
increasing insulin growth factor 1 levels in the ruminant with the feed ration; and
wherein the increase in insulin growth factor 1 levels supports skeletal development within the ruminant.

11. The method of claim 10, further comprising:
increasing concentration of hexoses within the rumen with the feed ration.

12. The method of claim 10, further comprising:
increasing rates of gluconeogenesis within the rumen with the feed ration.

13. The method of claim 10, further comprising:
increasing growth hormone levels within the ruminant with the feed ration

14. The method of claim 10, further comprising:
increasing height of the ruminant with the feed ration, wherein the ruminant is prepubescent.

15. The method of claim 10, further comprising:
decreasing a ratio of acetate to propionate within the rumen with the feed ration.

16. The method of claim 10, further comprising:
increasing dry matter intake of the ruminant with the feed ration.

17. A system for hydroponically sprouted cereal grains as a mechanism for improving skeletal development of a ruminant, the system comprising:
a grower for hydroponically sprouting one or more cereal grains for growing at least one feed component for prepubescent ruminants;
a feed ration having the at least one feed component comprising the one or more hydroponically sprouted cereal grains for improving skeletal development in the prepubescent ruminants.

18. The system of claim 17, wherein the feed ration having the at least one feed component comprising the one or more hydroponically sprouted cereal grains increases an average rate of daily gain in the prepubescent ruminants.

19. The system of claim 17, wherein the feed ration having the at least one feed component comprising the one or more hydroponically sprouted cereal grains increases insulin growth factor 1 in the prepubescent ruminants.

20. The system of claim 17, wherein the feed ration having the at least one feed component comprising the one or more hydroponically sprouted cereal grains increases leptin levels within the prepubescent ruminant.
